(19) United States
(12) Patent Application Publication
(10) Pub. No.: US 2024/0182951 A1
(43) Pub. Date: Jun. 6, 2024

(54) METHODS FOR TARGETED NUCLEIC ACID SEQUENCING

(71) Applicant: Jumpcode Genomics, Inc., San Diego, CA (US)

(72) Inventors: Keith BROWN, San Diego, CA (US); Jon ARMSTRONG, San Diego, CA (US); Azeem SIDDIQUE, San Diego, CA (US)

(21) Appl. No.: 18/553,167

(22) PCT Filed: Mar. 30, 2022
(86) PCT No.: PCT/US2022/022619

§ 371 Date: Sep. 28, 2023

Related U.S. Application Data
(60) Provisional application No. 63/168,831, filed on Mar. 31, 2021.

(51) Int. Cl. C12Q 1/6816; C12Q 1/44; C12Q 1/6806; C12Q 1/686; C12Q 1/6886

(52) U.S. Cl. CPC C12Q 1/6816; C12Q 1/44; C12Q 1/6806; C12Q 1/686; C12Q 1/6886

(57) ABSTRACT

Disclosed herein are compositions and methods related to the detection and/or elimination of a first nucleic acid and enrichment of a second nucleic acid in a sample, for example to exclude the first nucleic acid from downstream analysis or sequencing, or to exclude such sequences from a downstream data set.

### CROSS REFERENCE

This application claims the benefit of U.S. Provisional Application No. 62/168,831, filed on Mar. 31, 2021, which is incorporated herein by reference in its entirety.

### BACKGROUND

The disclosure herein relates to the field of molecular biology, such as methods and compositions for detecting, enriching and/or altering a target nucleic acid in a sample. The methods and compositions are applicable to biological, clinical, forensic, and environmental samples.

Cell free nucleic acid (cfNA) can be obtained from biological samples such as tissue, fluids, or other biological samples obtained from an organism, or forensic or archeological samples. CfNA is often of poor quality and can be available in limited quantity. Use of such nucleic acids for any downstream application requires amplification and preparation of nucleic acid libraries. However, existing methods are often time-consuming and inefficient. Therefore, there is a need for developing a more viable methods for preparation of cell free nucleic acid libraries and its downstream applications.

### SUMMARY

The instant application, in one aspect provides methods and compositions for obtaining nucleic acid regions or sequences from a sample that is available in low quality or low quantity or both, by precise and direct amplification from the source or origin, without prior isolation, purification, enrichment or amplification. In one aspect, provided herein is a method of detecting the presence or absence of a target nucleic acid from a sample comprising a plurality of nucleic acid molecules, the method comprising: contacting one or more nucleic acid molecules of the plurality of nucleic acid molecules with a synthetic nucleic acid comprising a first nucleic acid segment and a second nucleic acid segment that are in inverted orientation with respect to each other, thereby generating one or more synthetic circularized nucleic acid molecules; and sequencing the one or more synthetic circular nucleic acid molecules, thereby detecting the presence or absence of the target nucleic acid.

In one aspect, provided herein is a method of amplifying a target nucleic acid from a sample comprising a plurality of nucleic acid molecules, the method comprising: contacting one or more nucleic acid molecules of the plurality of nucleic acid molecules with a synthetic nucleic acid comprising a first nucleic acid segment and a second nucleic acid segment that are in inverted orientation from each other, wherein the one or more nucleic acid molecule comprises the target sequence, thereby generating one or more synthetic circularized nucleic acid molecules; and amplifying the one or more synthetic circularized nucleic acid molecules, thereby amplifying the target nucleic acid.

In one aspect, provided herein is a method of barcoding a plurality of nucleic acid molecules in a sample, the method comprising: contacting the plurality of nucleic acid molecules with a synthetic nucleic acid comprises a first nucleic acid segment and a second nucleic acid segment that are in inverted orientation from each other, wherein the first or the second nucleic acid segment comprises a molecular barcode, generating one or more synthetic circular circularized nucleic acid molecules; wherein each synthetic circularized nucleic acid molecules comprises a nucleotide barcoding embedded within the circularized nucleic acid molecules.

In some aspects, the synthetic nucleic acid is single stranded. In some aspects, the synthetic nucleic acid is double stranded, wherein the double stranded synthetic nucleic acid comprises single stranded regions.

In some aspects, the synthetic nucleic acid comprises a sequence having a configuration: 3′-X1m-A-5′-5′-B-X2n-3′ wherein X1m and X2n each denotes a sequence of m and n number of nucleotides respectively, wherein m and n each is any integer between 1 and 30, and A and B each represent any nucleotide, wherein A and B are juxtaposed in 5′-5′ inverted orientation. 3′-X1m-A-5′-5′-B-X2n-3′ comprises a single strand of nucleic acid. In some aspects, the single strand comprising 3′-X1m-A-5′-5′-B-X2n-3′ is DNA.

In some aspects, the synthetic nucleic acid comprises a sequence having a second strand (or a bottom strand) that comprises a sequence having a configuration: 3′-random sequence-1(RS1)-X3-random sequence-2 (RS2)-3′. In some aspects, at least a portion of X3 is complementary to a portion of the top strand (e.g., partially complementary to 3′-X1ₘ-A-5′-5′-B-X2ₙ-3′). In some aspects, the single strand comprising 3′-random sequence-1(RS1)-X3-random sequence-2 (RS2)-3′ is DNA. In some aspects, RS1 and RS2 each has at least 2, e.g., 3, 4, 5, 7, 8, 9, 10, 11, 12, 13, 14, 15 or more nucleotides that are single stranded at the 3′ terminus. RS1 and RS2 may be random adapter sequences.

In some aspects, the sample is a biological sample. In some aspects, the biological sample comprises low quantity of the plurality of nucleic acid molecules, or low quality of the plurality of nucleic acid molecules or both. In some aspects, the biological sample comprises cell free nucleic acid (cfNA). In some aspects, the biological sample comprises frozen nucleic acid. In some aspects, the biological sample comprises ancient nucleic acid. In some aspects, the plurality of nucleic acid molecules comprise DNA. In some aspects, the plurality of nucleic acid molecules comprise RNA. In some aspects, the plurality of nucleic acid molecules is a mixture of DNA and RNA. In some aspects, the plurality of nucleic acid molecules comprises single or double-stranded nucleic acid, or both.

In some aspects, the method further comprises a step of denaturing the plurality of nucleic acid molecules.

In some aspects, the method further comprises depleting one or more components of the plurality of nucleic acid molecules that is not bound to the synthetic nucleic acid. In some aspects, the method comprises selective depletion of unwanted DNA or RNA in the sample. Candidates for depletion, and hence enrichment of target nucleic acid comprises microbial contaminant nucleic acid, host's contaminant nucleic acid, abundant unwanted nucleic acid representing, based on the intended use, repeated nucleic acid sequences, or ribosomal RNA sequences. In some aspects, the depletion is performed before generation of a synthetic circularized nucleic acid molecules. In some aspects, the depletion is performed after generation of a synthetic circularized nucleic acid molecules. In some aspects, the depletion is performed using a nuclease. In some aspects, the nuclease is a DNA guided endonuclease. In some aspects, the nuclease is a DNA guided endonuclease is Argonaut (AGO). In some aspects, the nuclease is a CAS endonuclease.

In some aspects, the method further comprises annealing one or more adapter handles (short stretches of adapter nucleic acid sequences) to the synthetic nucleic acid.

In some aspects, an adapter handle is annealed to each termini of the single-stranded synthetic nucleic acid, the double stranded synthetic nucleic acid or a ligated product comprising the synthetic nucleic acid. In some aspects, an adapter handle comprises double stranded nucleic acid.

In some aspects, the method described herein further comprises performing polymerase chain reaction.

In some aspects, the method further comprises incorporating one or more modifications in the synthetic circularized nucleic acid molecules.

In some aspects, the method further comprises incorporating one or more modifications in the synthetic nucleic acid constructs or the synthetic circularized nucleic acid molecules. In some aspects, one or more modifications can be incorporated in the synthetic nucleic acid constructs (the single stranded top molecule or the single stranded bottom molecule, at the double stranded region of the molecule or in the random sequence of the bottom molecule). In some aspects, the method comprises incorporating a non-natural nucleotide, wherein the non-natural nucleotide is an LNA (locked nucleic acid) or a PNA (peptide nucleic acid). In some aspects, the method comprises incorporating one or more modifications comprises incorporating a non-canonical nucleotide backbone linkage at the ligation point. In some aspects, the non-canonical nucleotide backbone linkage comprises an amide linkage, a triazole linkage, or a phosphoramidate, e.g. at the junction between two inverted oligonucleotide sequences, such as the 5′-5′ nucleotide juxtaposition in the synthetic oligonucleotides. In some aspects, the ends of the synthetic polynucleotide are not phosphorylated.

Provided herein is a method for selectively enriching one or more target nucleic acids comprising the method steps of any of the aspects described above, wherein at least one or more nucleic acid components is depleted. In some aspects the one or more nucleic acid components that is depleted is contaminant nucleic acid, microbial nucleic acid, host nucleic acid, ribosomal RNA, or repeat nucleic acid.

In some aspects, the methods described herein are performed for diagnosing a disease. In some aspects, the disease is cancer. In some aspects, the disease is a microbial disease. In some aspects, the disease is a metabolic disease. In some aspects, the disease is genetic disease.

In some aspects, the method is performed for a microbiome analysis. In some aspects, the method is performed for non-invasive prenatal testing.

Provided herein is a synthetic single or double-stranded nucleic acid comprising an oligonucleotide having a configuration. 3′-X1m-A-5′-5′-B-X2n-3′ wherein X1m and X2n each denotes a sequence of m and n number of nucleotides respectively, wherein m and n depict any integer between 1 and 30, A and B each represent any nucleotide, wherein A and B are juxtaposed in 5′-5′ inverted orientation. In some aspects, the synthetic nucleic acid e.g., the synthetic polynucleotide is double stranded, and wherein the double stranded polynucleotide comprises single stranded regions. In some aspects, the single stranded regions within the double stranded polynucleotide comprise a sequence of 3 or more random nucleotides at the 5′ or the 3′ end of the double stranded region or both.

Provided herein is a nucleic acid library comprising the synthetic circularized nucleic acid molecule or portions thereof, or derivatives thereof of any one of the aspects described above.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIGS. 1A and 1B depict workflow of an exemplified amplification using stubby adapters.

FIGS. 2A and 2B depicts an exemplified strategy of one-step circularization without amplification.

FIG. 3 demonstrates rolling circle amplification and production of linear copies of the circular molecule. The cfNA inserts are between the adapters (P5 and P7) that are in the correct orientation.

### DETAILED DESCRIPTION

The instant disclosure is based at least in part on the need for an improved efficiency of single stranded library preparation for cell-free nucleic acids cfNAs. A majority of the cell free molecules are often degraded, not blunt ended, and much of the material is single-stranded. Traditional library preparation uses end repair, A-tailing and ligation of directional adapters. This is an inefficient process on cell-free material and often results in adapter dimers which are hard to separate from actual desired library material. The end result is often a significant loss in sensitivity and increase in cost due to the fact the artifacts generated will be sequenced. Ligation approaches also generate concatemers Thus, such procedures are not sensitive enough to analyze cell free materials and also incurs high costs from sequencing the artifacts generated. The instant disclosure attempts to resolve these issues and provide an efficient system for sequence library preparation from cell-free nucleic acid.

Disclosed herein are methods, systems, and compositions for efficient library preparation from cell-free nucleic acids, e.g., cell-free DNA or RNA. The resulting library can be sequenced, and can be utilized towards a large number of diagnostic and nucleic acid engineering applications.

The sequencing methods and systems utilize the consensus sequencing of the rolling circle amplified (RCA) short templates and nanopore sequencing techniques to provide a faster time to obtain sequencing results. The methods and systems work well with highly degraded templates and reduce polymerase errors using target specific (junction) primers during RCA. The amplification step described herein only amplifies copies of the template molecule and avoids the problem of amplifying copies of copies that will propagate polymerase errors during the first or early amplification cycles of a technique like PCR.

In some aspects, the methods and systems described herein utilize specially designed primers. In some aspects, the methods and systems described herein comprise designing primers that can be used universally. In some aspects, the methods and systems described herein comprise designing primers that can be used for site specific applications. In some aspects, the methods described herein utilize rolling circle amplification using the primer designs as described herein. As a result of practice of methods disclosed herein, one obtains a library that is both highly amplified, highly representative of the sample nucleic acid from very minute quantities of the original material, and/or from original nucleic acid materials that are degraded or damaged. Methods, compositions, and kits are provided for sequencing of targeted nucleic acids. These methods, compositions, and kits find use in a number of applications, such as point of care detection of time critical genomic information; infectious disease detection in humans, plants, and animals in real time and in remote locations; forensic DNA analysis; and microbiome detection.

In one aspect, the method described herein is a method for detection of a nucleic acid signature in a given biological sample, such as a mutation in the genome, or presence of a second genome or any part or fragment thereof, wherein the first genome referred to herein can be that of a host organism, e.g., a human genome, and the second genome can be that of a non-host organism, a pathogen, or a contaminant genome, as applicable to the sample or target for identification. The method allows for both DNA and RNA to be investigated. In some aspects, the library is a single stranded nucleic acid library.

Advances in genome sequencing technologies have greatly increased our understanding of human genetic variation and its contribution to disease. Short read DNA sequencing technologies (e.g., Illumina, Thermo Fisher, Qiagen) produce billions of short reads resulting in the routine identification of single nucleotide polymorphisms and small insertions and deletions. These short read sequencing technologies have not shown a sensitivity to detect more complex variation such as large scale chromosomal rearrangements, translocations, and mobile element rearrangements. These systems are also often expensive and require 24 hours or more to complete a sequencing run. Long read sequencing technologies (e.g., Pacific Biosciences, Oxford Nanopore) have shown the ability to generate single molecule read lengths in excess of 10,000 base pairs, but do not have the capacity to sequence and assemble a full human genome. Sequencing strategies disclosed herein can produce highly accurate consensus sequencing of the short read sequencers with the speed and portability of long read sequencers.

The disclosure herein relates to sequencing methods and systems that can produce highly accurate consensus sequencing with fast read speed and great portability. Some aspects relate to methods of consensus sequencing of rolling circle amplified short templates and method of preparing such templates.

Through practice of the disclosure herein, one can create nucleic acid sequence libraries from nucleic acid obtained from various sources, where the nucleic acid may have been degraded, damaged, obtained from difficult and rare sources, or in other words, difficult to amplify and create nucleic acid libraries from.

Through practice of the disclosure herein, one can selectively enrich nucleic acids of interest, or selectively deplete nucleic acids that are not of interest from a sample, and thus more accurately and efficiently detect non-host organism's genetic materials, pathogen, tumor, fetal DNA, alleles, and other nucleic acids of interest in a sample. Viewing pathogen detection as an example, whole genome sequencing, or shot gun sequencing, offers a promising solution to detect pathogens. A challenge can be that many sample types contain an abundance of host molecules, limiting the sensitivity of shot gun sequencing to detect non-host pathogen nucleic acids and increasing the amount of sequence that must be generated so as to obtain reads representative of rare molecules in the sample, such as molecules derived from a pathogen or other exogenous organism on a host derived nucleic acid sample. Pathogen detection can be used in a number of applications including, but not limited to, an infectious disease outbreak, detecting a pathogen in an immune compromised individual, detecting pathogens in a blood bank, detection of pathogens in veterinary or agricultural samples, detection of plant pathogens in agricultural samples, removal of bacterial contaminant from saliva samples, mitochondrial nucleic acid depletion, or chloroplast nucleic acid depletion. A similar challenge presents itself in the identification of any rare or single copy nucleic acid in a sample that also comprises high copy or non-interest nucleic acids.

A number of sample preparation approaches have been proposed to address these challenges. Differential lysis of cell types has been described. For example, human cells are lysed via one lysis method, DNA from those cells are degraded via exonuclease, then the remaining non-human cells are lysed and prepared for sequencing. Another method aims to degrade methylated DNA, more abundant in human DNA than pathogen DNA, has also been described. These approaches are specific to a particular cell type or nucleic acid modifications.

In one aspect, the methods and compositions described herein allow the ability to convert RNA and DNA into a library.

Provided herein are compositions and methods for selective target enrichment or selective background depletion that are readily performed on a broad range of samples and that do not require amplification for depletion.

In one aspect, provided herein is a method of detecting the presence or absence of a target nucleic acid from a sample comprising a plurality of nucleic acid molecules. In some aspects, the method comprises contacting one or more nucleic acid molecules of the plurality of nucleic acid molecules with a synthetic nucleic acid comprising a first nucleic acid segment and a second nucleic acid segment. In some aspects, the first and second nucleic acid segments are in inverted orientation from each other such that two segments can be used to generate one or more synthetic circularized nucleic acid molecules from the one or more nucleic acid molecule. As used herein, two nucleic acid molecules in inverted orientation refers any structure from which two nucleic acid polymerases (e.g., DNA polymerase) can extends two polynucleotide molecules independently to different directions. Thus, in some aspects, two nucleic acid segments in inverted orientation are located in a single-strand of nucleic acid molecule where 5′-ends of the two nucleic acid segments or 3′-ends of the nucleic acid segments are directly or indirectly coupled with each other. In some aspects, such generated synthetic circularized nucleic acid molecules are sequenced, thereby detecting the presence or absence of the target nucleic acid. In some aspects, the plurality of nucleic acid molecules may comprise cell free nucleic acid (cfNA), wherein the cfNA can be DNA or RNA, obtained from a biological sample. In some aspects, the plurality of nucleic acid molecules comprise single or double-stranded nucleic acid, or both. In some aspects, the cfNA is single stranded, double stranded or a mixture of both. In some aspects, the cfNA is 20-250 nucleobases long. In some aspects, the cfNA is 20-250 base pairs long. In some aspects, the cfNA is about 20, about 30, about 40, about 50, about 60, about 70, about 80, about 90, about 100 about 120, about 130, about 140, about 150, about 160, about 170, about 180, about 190 or about 200 base pairs, or longer in its length. In some aspects, the cfNA comprises 50-150 bases. In some aspects, the cfNA comprises 50-120 bases. In some aspects, cell free RNA may be isolated from vesicles.

In some aspects, the method generally provides isolation and detection of the presence or absence of a target nucleotide sequence from cfNA obtained from highly degraded or damaged sequences generated from poor quality source material. In some aspects, the cfNA is protected, e.g., via binding to protein, for example, histones or other DNA binding proteins such as transcription factors or regulators, or RNA binding proteins such as polymerases, ribosomal proteins. It is often encountered that poor quality nucleic acids comprise short fragments of about 50-120 bases, comprising both single and double stranded nucleotide sequences, nucleotide stretches with sticky ends, thus, can be highly problematic for direct amplification or detection of a specific sequence. Amplification by known techniques from such poor quality nucleic acid often lead to amplification of large quantity of junk material that greatly reduces the signal to noise ratio. In the method described herein, only nucleic acid that hybridizes to the synthetic sequences described below are preserved for downstream processes, whereas undesirably single-stranded circularized non-hybridized cfNAs are removed in the process, thus provides solution in that the background noise can be reduced greatly. In some aspects about 90%, about 80%, about 70%, about 60%, about 50%, about 40%, or about 30% of the noise is reduced by depletion of undesired materials. Additionally, the method prevents artifacts to be included in the sequencing library (e.g., artifacts from primer dimer formation, and self-ligation of single stranded overhang regions of a synthetic construct).

In some aspects, the methods described herein is used for identification of cell free markers for trauma, transplant rejection, internal wounds, cancer treatment etc. In some aspects, the identification can be performed either from RNA or DNA samples and of RNA or DNA targets.

In some aspects, the composition and methods described herein is used for identification transplantation rejections, to identify, for example, if donor components or the recipient components are subject to a specific response, which would indicate rejection or cell death of the donor organ.

In some aspects, the composition and methods described herein is useful in diagnostics in infectious diseases. Biological samples used may be blood, cerebrospinal fluid (CSF), serum, plasma, saliva, urine, feces, or mucus. and can be employed for detection of unknown pathogens after traditional testing has failed. In some aspects, the composition and methods described herein are useful in detection of pathogens/pathogenic markers from low amount of biological samples or non-invasively obtained samples such as oral samples. In some aspects, the composition and methods described herein are useful in detection of oral microbiome. In some aspects, the composition and methods described herein are useful in detection of blood microbiome.

In some aspects, the composition and methods described herein are used in identifying specific tumor mutations, resurgence or minimal residual disease, monitoring response to therapy. In some aspects, the composition and methods described herein can be utilized for detection of tumor cells or markers in low amount of biological sample, or from non-invasively obtained samples, such as nasal or oral swab sample.

In some aspects, detecting the presence or absence of a target nucleic acid comprises detecting the presence of a pathogenic sample in a host sample. In some aspects, detecting the presence or absence of a target nucleic acid comprises detecting the presence of a mutation in a biological sample. In some aspects, detecting the presence or absence of a target nucleic acid comprises detecting the presence of a methylated CpG sequence in the cfNA of a biological sample. In some aspects, detecting the presence or absence of a target nucleic acid comprises detecting the presence of a modified nucleotide in the cfNA or a biological sample, such as a methylated nucleotide. In some aspects, detecting the presence or absence of a target nucleic acid comprises presence of inserted genomic materials, such as long interspersed sequences (LINEs) and Alu sequences.

### Double Adaptors in Inverted Orientations

In some aspects, the synthetic nucleic acid comprises a primer or an adaptor. In some aspects, the synthetic nucleic acid comprises a set of primers or adapters, wherein the first nucleic acid segment includes a first primer or a first adapter; and the second nucleic acid segment includes a second primer or a second adapter. In some aspects, the first and second nucleic acid segments are in inverted orientation with respect to each other in a first nucleotide strand. For example, in some instances, the synthetic nucleic acid comprises a first strand comprising a first nucleic acid segment, wherein 5′-end of the first nucleic acid segment is juxtaposed with the 5′-end of a second nucleic acid segment on the first synthetic nucleic acid, giving rise to a synthetic polynucleotide molecule, 3′-X1ₘ-A-5′-5′-B-X2ₙ-3′ wherein X1ₘ and X2ₙ each denotes a sequence of m and n number of nucleotides respectively. In some aspects, m and n depict any integer between 1 and 30. In some aspects, A and B each represents any nucleotide. In some aspects, A and B are juxtaposed in 5′-5′ inverted orientation. In some aspects, A and B are coupled via a linker. In some cases, each of the 3′-X1ₘ-A-5′ and the 5′-B-X2ₙ-3′ is referred to as the “short stubby adapter” in the disclosure. The synthetic polynucleotide molecule, 3′-X1ₘ-A-5′-5′-B-X2ₙ-3′ is synthesized as a single polynucleotide strand (alternatively the X1ₘ-A-5′-5′-B-X2ₙ-3′ strand can be referred to as a single synthetic oligonucleotide strand, or the first synthetic oligonucleotide strand), that comprises at least one pair of nucleotides that are 5′-5′ juxtaposed, and at least two or more nucleotides that are bidirectional with respect to each other. In some cases the first synthetic oligonucleotide strand is referred to as the top strand in the disclosure. In some aspects, the single strand comprising 3′-X1m-A-5′-5′-B-X2n-3′ is DNA.

In some aspects, the synthetic nucleic acid comprises a second strand. In some aspects, the second strand comprises nucleic acid sequence that is at least partially complementary to the first synthetic oligonucleotide, having the structure 3′-X1ₘ-A-5′-5′-B-X2ₙ-3′. Thus, in some aspects, the first and second synthetic oligonucleotide strands at least partially form a double-stranded nucleic acid (often referred to as polynucleotide comprising a double stranded sequence or a partially double stranded sequence). In some instances, the first and/or second strands include one or more random nucleotides at either end (either 5′-end or 3′-end). In some cases, the first synthetic oligonucleotide strand is referred to as the bottom strand in the disclosure. In some aspects, the bottom strand pairs with the top strand by Watson-Crick nucleobase pairing, and in addition, the one or more random nucleotides (or random sequences (RS)) at either end do not exhibit pairing with the top strand by Watson-Crick nucleobase pairing. In some aspects, the bottom strand includes the structure 3′-random sequence-1(RS1)-X3-random sequence-2 (RS2)-3′. In some aspects, at least a portion of X3 is complementary to a portion of the top strand (e.g., partially complementary to 3′-X1ₘ-A-5′-5′-B-X2ₙ-3′). In some aspects, X3 is at least 50%, at least 60%, at least 70%, at least 80%, at least 90% complementary to at least 50% of, at least 60% of, at least 70% of, at least 80% of, at least 90% of 3′-X1ₘ-A-5′-5′-B-X2ₙ-3′. RS1 represents random sequence-1, represented by one or more random nucleotides at one end of the fragment, and RS2 represents random sequence-2, represented by one or more random nucleotides at the other end of the fragment. In some aspects, at least 70%, at least 80%, at least 90% of RS1 or RS2 does not pair with the top strand such that at least a part of the RS1 or RS2 is available to bind to a portion of cfNA. In some aspects, neither RS1 nor RS2 can pair with any part of the top strand. In some aspects, the RS1 and/or RS2 are represented as overhangs of the synthetic single stranded molecule. In some aspects, the one or more nucleotides of the RS1 and the RS2 comprises about 12 nucleotides. The first strand and the second strand comprise a double stranded synthetic molecule, having the double stranded structure 3′-X1ₘ-A-5′-5′-B-X2ₙ-3′, wherein the first strand (or top strand, or top construct) and the second strand (or bottom strand or bottom construct) are paired by Watson-Crick nucleotide base pairing, and single stranded regions at either end, e.g., the RS1 and RS2 regions. In some cases, the RS1 and the RS2 sequences are termed as adapters in the disclosure.

In some aspects, the synthetic polynucleotide of the disclosure comprises an (i) upper strand, comprising two oligonucleotides that are directed outwards and ligated at one end, comprising a sequence that is denoted by the symbol, 3′-X1ₘ-A-5′-5′-B-X2ₙ-3′, as described in the previous paragraph; and (ii) a lower strand having a sequence 3′-RS1-5′-X3-5′-RS2-3′. In one aspect, a portion or X3 hybridizes with a portion of X1ₘ and/or A (e.g., an inner portion, towards the center of the upper strand) and a portion of X3 hybridizes with a portion of X2ₙ and/or B (e.g., an inner portion, towards the center of the upper strand). In some aspects a portion of RS1 hybridizes with a portion of X1ₘ (e.g., an outer portion, away from the center of the upper strand) and a portion of RS2 hybridizes with a portion of X2ₙ (e.g., an outer portion, away from the center of the upper strand).

In some aspects, the 3′-RS1-X1ₘ-A-5′ and the 5′-B-X2ₙ-RS2-3′ are of the same length. In some aspects the 3′-RS1-X1ₘ-A-5′ and the 5′-B-X2ₙ-RS2-3′ each is 24-50 bases long. In some aspects the 3′-RS1-X1ₘ-A-5′ and the 5′-B-X2ₙ-RS2-3′ each is 24 nucleotides long. In some aspects the 3′-RS1-X1ₘ-A-5′ and the 5′-B-X2ₙ-RS2-3′ each is 25 nucleotides long. In some aspects the 3′-RS1-X1ₘ-A-5′ and the 5′-B-X2ₙ-RS2-3′ each is 26 nucleotides long. In some aspects the 3′-RS1-X1ₘ-A-5′ and the 5′-B-X2ₙ-RS2-3′ each is 27 nucleotides long. In some aspects the 3′-RS1-X1ₘ-A-5′ and the 5′-B-X2ₙ-RS2-3′ each is 28 nucleotides long. In some aspects the 3′-RS1-X1m-A-5′ and the 5′-B-X2ₙ-RS2-3′ each is 29 nucleotides long. In some aspects the 3′-RS1-X1m-A-5′ and the 5′-B-X2n-RS2-3′ each is 30 nucleotides long. In some aspects the 3′-RS1-X1m-A-5′ and the 5′-B-X2n-RS2-3′ each is 31 nucleotides long. In some aspects the 3′-RS1-X1m-A-5′ and the 5′-B-X2n-RS2-3′ each is 32 nucleotides long. In some aspects the 3′-RS1-X1m-A-5′ and the 5′-B-X2n-RS2-3′ each is 33 nucleotides long. In some aspects the 3′-RS1-X1m-A-5′ and the 5′-B-X2n-RS2-3′ each is 34 nucleotides long. In some aspects the 3′-RS1-X1ₘ-A-5′ and the 5′-B-X2ₙ-RS2-3′ each is 35 nucleotides long. In some aspects the 3′-RS1-X1m-A-5′ and the 5′-B-X2n-RS2-3′ each is 36 nucleotides long. In some aspects the 3′-RS1-X1m-A-5′ and the 5′-B-X2n-RS2-3′ each is 37 nucleotides long. In some aspects the 3′-RS1-X1m-A-5′ and the 5′-B-X2n-RS2-3′ each is 38 nucleotides long. In some aspects the 3′-RS1-X1m-A-5′ and the 5′-B-X2n-RS2-3′ each is 39 nucleotides long. In some aspects the 3′-RS1-X1m-A-5′ and the 5′-B-X2n-RS2-3′ each is 40 nucleotides long. In some aspects the 3′-RS1-X1m-A-5′ and the 5′-B-X2n-RS2-3′ each is 41 nucleotides long. In some aspects the 3′-RS1-X1m-A-5′ and the 5′-B-X2n-RS2-3′ each is 42 nucleotides long. In some aspects the 3′-RS1-X1m-A-5′ and the 5′-B-X2n-RS2-3′ each is 43 nucleotides long. In some aspects the 3′-RS1-X1m-A-5′ and the 5′-B-X2n-RS2-3′ each is 44 nucleotides long. In some aspects the 3′-RS1-X1m-A-5′ and the 5′-B-X2n-RS2-3′ each is 45 nucleotides long. In some aspects the 3′-RS1-X1m-A-5′ and the 5′-B-X2n-RS2-3′ each is 46 nucleotides long. In some aspects the 3′-RS1-X1m-A-5′ and the 5′-B-X2n-RS2-3′ each is 47 nucleotides long. In some aspects the 3′-RS1-X1m-A-5′ and the 5′-B-X2n-RS2-3′ each is 48 nucleotides long. In some aspects the 3′-RS1-X1m-A-5′ and the 5′-B-X2n-RS2-3′ each is 49 nucleotides long. In some aspects the 3′-RS1-X1m-A-5′ and the 5′-B-X2n-RS2-3′ each is 50 nucleotides long.

In some aspects, the 3′-RS1-X1m-A-5′ and the 5′-B-X2n-RS2-3′ are in different lengths. For example, in some aspects, 3′-RS1-X1ₘ-A-5′ is from about 15 to about 50 bases long. In some aspects, 5′-B-X2ₙ-RS2-3′ is from about 15 to about 50 bases long. In some aspects, 3′-RS1-X1m-A-5′ is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 bases longer or shorter than 5′-B-X2n-RS2-3′.

In some aspects the 5′ and 3′ ends of the bottom construct is capped to avoid any ligation events.

In one aspect, the synthetic construct described herein is utilized to generate a rolling circle product comprising a sequence from a biological sample, such as a cfNA. In some aspects, the synthetic construct is contacted with nucleic acid from a biological sample, e.g., cell free nucleic acid (cfNA), such as frozen nucleic acid or FFPE sample, wherein the random nucleotide adapters e.g., at the RS1 and RS2 hybridize with the cfNA. The cfNA is denatured prior to contacting with the synthetic construct. The template single stranded cfNA is hybridized to the random sequences on the bottom construct. For example, one end of the single stranded cfNA hybridizes (binds) to RS1 and another end of the single-stranded cfNA hybridizes (binds) to RS2 such that the cfNA and the synthetic construct form a circular nucleic acid. The ligation thereafter produces a circular product with the inverted adapters from the synthetic adapter (top, below) forming a closed circle.

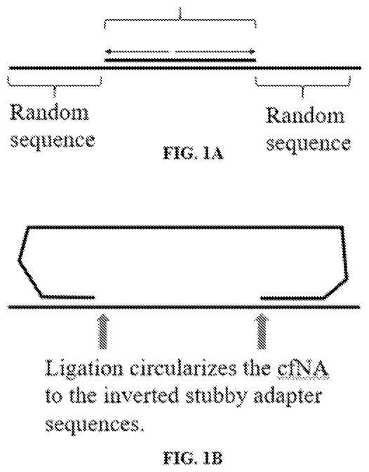

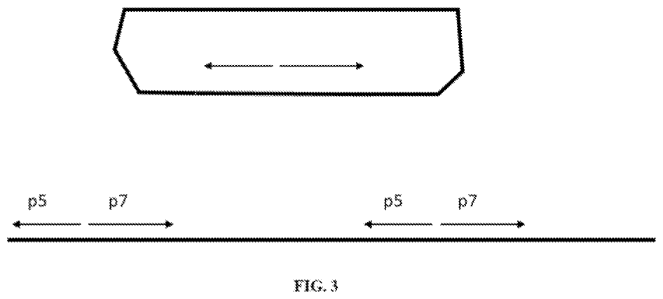

FIGS. 1A and 1B are schematic diagrams that generally exemplifies the formation of circularized cfNA product using the synthetic polynucleotide sequences described above. Rolling circle amplification produces linear copies of the circular molecules (FIG. 3). The cfNA inserts are between the adapters that are in correct orientation. PCR of template with full length adapters include sample barcodes and UMIs.

In some aspects, the circular products are used to generate a library of short sequences from a biological sample. In some aspects, the rolling circle amplification is used to amplify and make a linear construct from the circular templates with the adapters now in the proper orientation for PCR based enrichment and library generation.

In some aspects, the undesired circularized single stranded molecules are cleaved and digested by an exonuclease to remove the strands that do not generate as a result of hybridizing to the RS1 or RS2 ends. Argonaute or other specific endonucleases can be used for clipping open the undesired circular constructs. Argonaute is a DNA guided endonuclease for site-specific cleavage and digestion.

In some aspects, preparation of the rolling circle product comprising a fragment of a nucleic acid from a biological sample and generation of a library of sequences from the biological sample comprises the following steps:
Isolation of cfNA
Denaturation of the cfNA
Hybridization to the synthetic construct
Ligation
Nuclease (e.g. Argonaute) digestion (or endonuclease cleavage at sequence specific sites) of overabundant/uninformative templates
Exonuclease digestion of linear templates
Rolling circle amplification
Low cycle PCR and sample barcoding
Size selection/cleanup
Load flow cell (sequencer) for cluster generation and sequencing
Data analysis

In some aspects, preparation of the rolling circle product comprising a fragment of a nucleic acid from a biological sample and generation of a library of sequences from the biological sample comprises the following steps:
Isolation of cfNA
Denaturation of the cfNA
Hybridization to the synthetic construct
Ligation
Nuclease digestion (endonuclease cleavage at sequence specific sites) of overabundant/uninformative templates
Rolling circle amplification
Exonuclease digestion of linear templates
Low cycle PCR and sample barcoding
Size selection/cleanup
Load flow cell (sequencer) for cluster generation and sequencing
Data analysis

In one aspect, provided herein is a method of amplifying a target nucleic acid from a sample comprising a plurality of nucleic acid molecules, the method comprising: contacting one or more nucleic acid molecules of the plurality of nucleic acid molecules with a synthetic nucleic acid comprising a first nucleic acid segment and a second nucleic acid segment that are in inverted orientation from each other, wherein the one or more nucleic acid molecule comprises the target sequence, thereby generating one or more synthetic circularized nucleic acid molecules; and amplifying the one or more synthetic circularized nucleic acid molecules, thereby amplifying the target nucleic acid.

In some aspects, the synthetic nucleic acid comprises a first strand comprising a first nucleic acid segment, wherein 5′-end of the first nucleic acid segment is juxtaposed with the 5′-end of a second nucleic acid segment on the first synthetic nucleic acid, giving rise to a synthetic polynucleotide molecule, 3′-X1ₘ-A-5′-5′-B-X2ₙ-3′ wherein X1ₘ and X2ₙ each denotes a sequence of m and n number of nucleotides respectively, wherein m and n depict any integer between 1 and 30, A and B each represent any nucleotide, wherein A and B are juxtaposed in 5′-5′ inverted orientation. The synthetic polynucleotide molecule, 3′-X1ₘ-A-5′-5′-B-X2ₙ-3′ is synthesized as a single polynucleotide strand (alternatively the X1ₘ-A-5′-5′-B-X2ₙ-3′ strand is referred to as a single synthetic oligonucleotide strand, or the first synthetic oligonucleotide strand), that comprises at least one pair of nucleotides that are 5′-5′ juxtaposed, and at least two or more nucleotides that are bidirectional with respect to each other. In some cases the first synthetic oligonucleotide strand is referred to as the top strand in the disclosure.

In some aspects, the synthetic nucleic acid comprises a second strand. In some aspects, the second strand comprises nucleic acid sequence that is at least partially complementary to the first synthetic oligonucleotide, having the structure 3′-X1ₘ-A-5′-5′-B-X2ₙ-3′. Thus, in some aspects, the first and second synthetic oligonucleotide strands at least partially form a double-stranded nucleotide. In some instances, the first and/or second strands include one or more random nucleotides at either end (either 5′-end or 3′-end). In some cases, the first synthetic oligonucleotide strand is referred to as the bottom strand in the disclosure. In some aspects, the bottom strand pairs with the top strand by Watson-Crick nucleobase pairing, and in addition, the one or more random nucleotides (or random sequences (RS)) at either end do not exhibit pairing with the top strand by Watson-Crick nucleobase pairing. In some aspects, the bottom strand includes the structure 3′-random sequence-1(RS1)-X3-random sequence-2 (RS2)-3′. In some aspects, at least a portion of X3 is complementary to a portion of the top strand (e.g., partially complementary to 3′-X1ₘ-A-5′-5′-B-X2ₙ-3′). In some aspects, X3 is at least 50%, at least 60%, at least 70%, at least 80%, at least 90% complementary to at least 50% of, at least 60% of, at least 70% of, at least 80% of, at least 90% of 3′-X1ₘ-A-5′-5′-B-X2ₙ-3′.

In some aspects, the synthetic polynucleotide comprises a partially double stranded DNA comprising an (i) upper strand, comprising two oligonucleotides that are directed outwards and ligated at one end, comprising a sequence that is denoted by the symbol, 3′-X1ₘ-A-5′-5′-B-X2ₙ-3′, as described in the previous paragraph; and (ii) a lower strand having a sequence 3′-RS1-5′-X3-5′-RS2-3′. In one aspect, a portion or X3 hybridizes with the nucleotides through the entire length of or a portion of X1ₘ and/or A (e.g., an inner portion, towards the center of the upper strand) and a portion of X3 hybridizes with the nucleotides through the entire length of or a portion of X2ₙ and/or B (e.g., an inner portion, towards the center of the upper strand).

In some aspects, X3 exhibits Watson Crick pairing with at least 2, or 3, 4, 5, 6, 7, 8, 9, 10, 11, 12 or more contiguous nucleotides of 3′-X1ₘ-A-5′; wherein m is at least greater than at least 3, 4, 5, 6, 7, 8, 9, 10, 11, 12, 13 or more nucleotides respectively. In some aspects, X3 exhibits Watson Crick pairing with at least 2, or 3, 4, 5, 6, 7, 8, 9, 10, 11, 12 or more contiguous nucleotides of 5′-B-X2n-3′; wherein n is at least greater than at least 3, 4, 5, 6, 7, 8, 9, 10, 11, 12, 13 or more nucleotides respectively.

In some aspects, the first and second oligonucleotide strands (3′-X1ₘ-A-5′-5′-B-X2ₙ-3′) are prepared synthetically.

In some aspects, the lower strand (3′-RS1-5′-X3-5′-RS2-3′) is a synthetic oligonucleotide.

In some aspects, the first and second synthetic oligonucleotide strands comprise a primer pair sequence. In some aspects the first synthetic oligonucleotide strands comprises a sequence of a first primer of a primer pair sequence, or a sequence complementary to a first primer sequence. In some aspects the second synthetic oligonucleotide strand comprises a sequence of a second primer of a primer pair sequence, or a sequence complementary to a second primer sequence. In some aspects, as described in the paragraphs above, the primer pair sequences comprised within 3′-X1ₘ-A-5′ and 5′-B-X2ₙ-3′ are bidirectional, on a single strand, radiate in opposite directions. In some aspects, the primer pair comprises at least one nucleotide each that are 5′-5′ juxtaposed with each other, denoted as A-5′-5′-B, as described above. Similarly, the lower strand comprises sequences complementary to the first primer sequence and the second primer sequence. In some aspects the lower strand comprises at least two adjacent nucleotides that are 5′-5′ juxtaposed with each other. In some aspects the lower strand comprises at least two adjacent nucleotides that are 5′-5′ juxtaposed with each other that are complementary to A-5′-5′-B of the first and second oligonucleotide strands (3′-X1ₘ-A-5′-5′-B-X2ₙ-3′).

RS1 represents random sequence-1, represented by one or more random nucleotides at one end of the fragment, and RS2 represents random sequence-2, represented by one or more random nucleotides at the other end of the fragment. In some aspects, at least 70%, at least 80%, at least 90% of RS1 or RS2 does not pair with the top strand such that at least a part of the RS1 or RS2 is available to bind to a portion of cfNA. In some aspects, neither RS1 nor RS2 can pair with any part of the top strand. In some aspects, the RS1 and/or RS2 are represented as overhangs of the synthetic double stranded molecule that is comprised of the paired top strand and bottom strand.

In some aspects, the one or more nucleotides of the RS1 and the one or more nucleotides of the RS2 each comprises about 12 nucleotides. The first strand and the second strand comprise a double stranded synthetic molecule (e.g., a synthetic polynucleotide), having the double stranded structure 3′-X1ₘ-A-5′-5′-B-X2ₙ-3′, wherein the first strand (or top strand, or top construct) and the second strand (or bottom strand or bottom construct) are paired by Watson-Crick nucleotide base pairing, and single stranded regions at either end, e.g., the RS1 and RS2 regions. In some cases, the RS1 and the RS2 sequences are termed as adapters in the disclosure.

In some aspects the 5′ and 3′ ends of the bottom construct should be capped to avoid any ligation events.

In one aspect, the synthetic construct described herein is utilized to generate a rolling circle product comprising a sequence from a biological sample, such as a cfNA. In some aspects, the synthetic construct is contacted with nucleic acid from a biological sample, e.g., cell free nucleic acid (cfNA), such as frozen nucleic acid or FFPE sample, wherein the random nucleotide adapters e.g., at the RS1 and RS2 hybridize with the cfNA. The cfNA is denatured prior to contacting with the synthetic construct. The template single stranded cfNA is hybridized to the random sequences on the bottom construct. For example, one end of the single stranded cfNA hybridizes (binds) to RS1 and another end of the single-stranded cfNA hybridizes (binds) to RS2 such that the cfNA and the synthetic construct form a circular nucleic acid. The ligation thereafter produces a circular product with the inverted adapters from the synthetic adapter (top, below) forming a closed circle.

In some aspects, the circular products are used to amplify a desired sequence in the cfNA, or universally, the sequences in the cfNA captured in the rolling circle by PCR based amplification with desired primers.

In one aspect, provided herein is a method of barcoding a plurality of nucleic acid molecules in a sample, the method comprising contacting the plurality of nucleic acid molecules with an a synthetic nucleic acid comprises a first nucleic acid segment and a second nucleic acid segment that are in inverted orientation from each other, wherein the first or the second nucleic acid segment comprises a molecular barcode, generating one or more synthetic circular circularized nucleic acid molecules; wherein each synthetic circularized nucleic acid molecules comprises a nucleotide barcoding embedded within the circularized nucleic acid molecules.

For example, in some instances, the synthetic nucleic acid comprises a first strand comprising a first nucleic acid segment, wherein 5′-end of the first nucleic acid segment is juxtaposed with the 5′-end of a second nucleic acid segment on the first synthetic nucleic acid, giving rise to a synthetic polynucleotide molecule, 3′-X1ₘ-A-5′-5′-B-X2ₙ-3′ wherein X1m and X2n each denotes a sequence of m and n number of nucleotides respectively. In some aspects, m and n depict any integer between 1 and 30. In some aspects, A and B each represents any nucleotide. In some aspects, A and B are juxtaposed in 5′-5′ inverted orientation. In some aspects, A and B are coupled via a linker. In some cases, each of the 3′-X1ₘ-A-5′ and the 5′-B-X2ₙ-3′ is referred to as the “short stubby adapter” in the disclosure. The synthetic polynucleotide molecule, 3′-X1ₘ-A-5′-5′-B-X2ₙ-3′ is synthesized as a single polynucleotide strand (alternatively the X1ₘ-A-5′-5′-B-X2ₙ-3′ strand can be referred to as a single synthetic oligonucleotide strand, or the first synthetic oligonucleotide strand), that comprises at least one pair of nucleotides that are 5′-5′ juxtaposed, and at least two or more nucleotides that are bidirectional with respect to each other. In some cases the first synthetic oligonucleotide strand is referred to as the top strand in the disclosure.

In some aspects, the synthetic nucleic acid comprises a second strand. In some aspects, the second strand comprises nucleic acid sequence that is at least partially complementary to the first synthetic oligonucleotide, having the structure 3′-X1ₘ-A-5′-5′-B-X2ₙ-3′. Thus, in some aspects, the first and second synthetic oligonucleotide strands at least partially form a double-stranded nucleotide. In some instances, the first and/or second strands include one or more random nucleotides at either end (either 5′-end or 3′-end). In some cases, the first synthetic oligonucleotide strand is referred to as the bottom strand in the disclosure. In some aspects, the bottom strand pairs with the top strand by Watson-Crick nucleobase pairing, and in addition, the one or more random nucleotides (or random sequences (RS)) at either end do not exhibit pairing with the top strand by Watson-Crick nucleobase pairing. In some aspects, the bottom strand includes the structure 3′-random sequence-1(RS1)-X3-random sequence-2 (RS2)-3′. In some aspects, at least a portion of X3 is complementary to a portion of the top strand (e.g., partially complementary to 3′-X1ₘ-A-5′-5′-B-X2ₙ-3′). In some aspects, X3 is at least 50%, at least 60%, at least 700, at least 80%, at least 90% complementary to at least 50% of, at least 60% of, at least 70% of, at least 80% of, at least 90% of 3′-X1ₘ-A-5′-5′-B-X2ₙ-3′. RS1 represents random sequence-1, represented by one or more random nucleotides at one end of the fragment, and RS2 represents random sequence-2, represented by one or more random nucleotides at the other end of the fragment. In some aspects, at least 70%, at least 80%, at least 90% of RS1 or RS2 does not pair with the top strand such that at least a part of the RS1 or RS2 is available to bind to a portion of cfNA. In some aspects, neither RS1 nor RS2 can pair with any part of the top strand. In some aspects, the RS1 and/or RS2 are represented as overhangs of the synthetic double stranded molecule that is comprised of the paired top strand and bottom strand. In some aspects, the one or more nucleotides of the RS1 and the RS2 comprises about 12 nucleotides. The first strand and the second strand comprise a double stranded synthetic molecule, having the double stranded structure 3′-X1ₘ-A-5′-5′-B-X2ₙ-3′, wherein the first strand (or top strand, or top construct) and the second strand (or bottom strand or bottom construct) are paired by Watson-Crick nucleotide base pairing, and single stranded regions at either end, e.g., the RS1 and RS2 regions. In some cases, the RS1 and the RS2 sequences are termed as adapters in the disclosure.

In some aspects, the 5′ and 3′ ends of the bottom construct should be capped to avoid any ligation events.

In one or more aspects disclosed herein, the synthetic double stranded oligonucleotides having the structure 3′-RS1-X1m-A-5′-5′-B-X2n-RS2-3′, comprises a nucleotide barcode, such that once the circularized product is formed the barcode is embedded in a circularized product. In some aspects, the nucleotide barcode is present within the structure of 3′-X1ₘ-A-5′-5′-B-X2ₙ-3′. In some aspects, a nucleotide barcode is a unique sequence of nucleotides that is required to further identify any nucleotide composition that comprises the unique sequence of nucleotides.

In some aspects, the synthetic nucleic acid is single stranded.

In some aspects, the synthetic nucleic acid is double stranded, wherein the double stranded synthetic nucleic acid comprises single stranded regions (e.g., overhangs).

In one aspect, creating a circular library construct from cfNAs allows one to degrade any adapter dimer artifacts and potentially isolation of cell free (usually 50-120 bp) from just additional cellular nucleic acids that may have been exposed during the sample preparation or extraction.

In some aspects, the method described herein can be used to deplete background nucleic acid sequences and enrich the sequences that are desired. For example, when looking for cell free nucleic acids representing pathogens, there can be human (host) nucleic acid as background noise, which would have to be removed. In some aspects, e.g., in an application where specific cancer mutations are searched for, removal of wild type nucleic acid can be desired. In some aspects, when looking for trauma signatures in a human biological sample, for example, there is an abundance of human ribosomal sequences, masking the sensitivity for the transcripts that would otherwise indicate some sort of cellular stress. In such conditions, it is desirable to remove the excess unwanted nucleic acid materials, and the method discussed in the previous sections is utilized as is clear to one of skill in the art. For example, a method to deplete unwanted nucleic acid sequences from a sample can employ the following functional steps:
Isolate cell free nucleic acids,
Denature cfNA to insure majority are single stranded,
Form the circular ligation product,
Exonuclease digest the other strand and unligated, uncircularized nucleic acid strands,
Rolling circle amplify and Argonaute deplete (or Argonaute deplete and then rolling circle amplify).

In some aspects, the rolling circle products generate linear templates where the first segment and the second segment of the synthetic sequences comprising 3′-X1ₘ-A-5′-5′-B-X2ₙ-3′ (that are in inverted orientation) on the circular template can produce inserts flanked by the 3′-X1ₘ-A-5′ and the 5′-B-X2ₙ-3′ templates in the proper orientation so that low cycle PCR can incorporate the full length adapter sequences. Thus only targets of interest can be converted into a sequencing library. The process provides greater efficiency, lower inputs, uniform representation (linear amplification) reduced artifacts and enriched signal for desired molecules.

In some aspects, 3′-X1ₘ-A-5′ and 5′-B-X2ₙ-3′ are nucleic acid sequences derived from known oligomers, e.g., a priori known primer sequence. Exemplary primer sequences includes, but not limited to, Illumina primer sequences P5 and P7. In some aspects, the 3′-X1ₘ-A-5′ and 5′-B-X2ₙ-3′ comprises a barcode sequence. In some aspects, RS1 and/or RS2 comprises at least a sequence that is desired to be amplified from a cfNA. In some aspects, RS1 and/or RS2 overhang regions comprises target specific regions, for example, a sequence that is expected to be present in the cfNA, such that target specific hybridization and amplification occurs from a cfNA. The 3′-X1ₘ-A-5′ and 5′-B-X2ₙ-3′ are synthesized in inverted orientation. Both the top and bottom strand are synthetic.

Denaturing cfNA ensures uniformity in the starting material for the method described herein. Nucleic acid obtained from sources such as FFPE sample, or frozen samples or archeological samples may comprise a heterogenous composition where some of the molecules are single stranded, some double stranded, some partially single and double stranded. Denaturing ensures that all template or target cfNAs are single stranded, enabling the hybridization to the random sequences from the synthetic construct. Denaturing can be achieved by any process known to one of skill in the art, such as heat denaturation, or chemical denaturation.

Provided herein are synthetic single or double-stranded nucleic acid comprising an oligonucleotide having a configuration: 3′-X1ₘ-A-5′-5′-B-X2ₙ-3′ wherein X1ₘ and X2ₙ each denotes a sequence of m and n number of nucleotides respectively, wherein m and n depict any integer between 1 and 30, A and B each represent any nucleotide, wherein A and B are juxtaposed in 5′-5′ inverted orientation. In some aspects, the m and the n comprise the same number of nucleotides. In some aspects the m or n comprise 5-100 nucleotides each.

In some aspects the synthetic polynucleotide is double stranded, and wherein the double stranded polynucleotide comprises single stranded regions.

In some aspects, an adapter handle is annealed to each termini of the single-stranded synthetic nucleic acid, the double stranded synthetic nucleic acid or a ligated product comprising the synthetic nucleic acid.

In some aspects, the single stranded regions within the double stranded polynucleotide comprise a sequence of 3 or more random nucleotides at the 5′ or the 3′ end of the double stranded region or both. The random nucleotide sequences, such as RS1 and RS2 sequences described above is 4, 5, 6, 7, 8, 9, 10, 11, 12, 13, 14, 15, 16, 17, 18, 19, 20, 21, 22, 23, 24, 25, 26, 27, 28, 29 or 30 nucleotides long. In some aspects, RS1 and/or RS2 is more than 30 nucleotides long. In some aspects, each of the RS1 and/or RS2 sequences is 5-25 nucleotide long random sequences. In some aspects, RS1 and/or RS2 sequences is 6-25 nucleotide long random sequences. In some aspects, RS1 and/or RS2 sequences is 7-25 nucleotide long. In some aspects RS1 and/or RS2 sequences is about 9-25 nucleotide long. In some aspects, RS1 and/or RS2 sequences is about 8-25 nucleotide long. In some aspects, RS1 and/or RS2 sequences is about 10-25 nucleotide long. In some aspects, RS1 and/or RS2 sequences is about 5-24 nucleotide long. In some aspects, RS1 and/or RS2 sequences is about 5-23 nucleotide long. In some aspects, RS1 and/or RS2 sequences is about 5-22 nucleotide long. In some aspects, RS1 and RS2 sequences is about 5-21 nucleotide long. In some aspects, RS1 and/or RS2 sequences is about 5-20 nucleotide long. In some aspects, RS1 and/or RS2 sequences is about 5-19 nucleotide long. In some aspects, RS1 and/or RS2 sequences is about 5-18 nucleotide long. In some aspects, RS1 and/or RS2 sequences is about 5-15 nucleotide long. In some aspects, RS1 and/or RS2 sequences is about 8-20 nucleotide long and comprise the capturing end for capturing and hybridizing sequences of the cfNA.

In some aspects each of the short stubby adapter sequences is up to about 60 bases in length. In some aspects, each of the short stubby adapter sequences is about 10-60, 10-59, 10-58, 10-57, 10-56, 10-55, 10-54, 10-53, 10-52, 10-51, 10-50, 10-49,10-48, 10-47,10-46, 10-45, 10-44, 10-43, 10-42, 10-41, 10-40, 10-39, 10-38, 10-37, 10-36, 10-35, 10-34, 10-33, 10-32, 10-31,10-30, 10-29, 10-28, 10-27, 10-26, 10-25, 10-24, 10-23, or 10-32, nucleotides long. In some aspects, each of the short stubby adapter sequences is about 15-about 30 nucleotides long. In some aspects, each of the short stubby adapter sequences is 15-30 nucleotides long. In some aspects, the short stubby adapter sequences is 18-27 nucleotides long.

In some aspects, a cyclic nucleotide (such as a phosphoramidite) is used at the two nucleotides juxtaposed at 5′ and 5′ ends of the synthetic construct (e.g., the “top” strand). This is used to mitigate the effect of secondary structures of circular constructs that could inhibit rolling circle amplification. This is optional, but potentially a useful addition to the construct.

In some aspects, the method further comprises incorporating one or more modifications in the synthetic nucleic acid constructs or the synthetic circularized nucleic acid molecules. In some aspects, one or more modifications are incorporated in the synthetic nucleic acid constructs (the single stranded top molecule or the single stranded bottom molecule, at the double stranded region of the molecule or in the random sequence of the bottom molecule). In some aspects the method comprises incorporating a non-natural nucleotide, wherein the non-natural nucleotide is an LNA or a PNA. In some aspects the method comprises incorporating one or more modifications comprises incorporating a non-canonical nucleotide backbone linkage at the ligation point. In some aspects the non-canonical nucleotide backbone linkage comprises an amide linkage, a triazole linkage, or a phosphoramidate. For the generation of cyclic intermediates the cyclic templates containing a phosphoramidate linkage were particularly well tolerated by ϕ-29 polymerase, consistently performing as well in RCA as the unmodified DNA controls. Additionally, phosphoramidate-modified cyclic constructs can be readily produced in oligonucleotide synthesis facilities from commercially available precursors. Phosphoramidate ligation is therefore a practical and scalable method for the synthesis of cyclic RCA templates. The triazole-modified cyclic templates tend to produce lower and more variable yields of RCA products, a significant proportion of which were double-stranded, while the performances of the templates containing an amide linkage lie in between those of the phosphoramidate- and triazole-containing templates. In some aspects, the ends of the synthetic polynucleotide are not phosphorylated. In some aspects, one of the synthetic strand that functions as a template for the rolling circle amplification may comprise an Uracil residue. The Uracil residue may further be degraded using UDG/APOE or USER

In some aspects, a protein induced DNA bending can be utilized to assist the binding of an oligonucleotide (e.g., of a cfNA strand) to the one or more primers or adapters described, for example to RS1 and/or RS2. Several DNA binding proteins can be implicated in inducing conformational changes such as bending. An exemplary protein that induces DNA bending can be integration host factor (IHF). Virally encoded Int and Xis proteins and the bacterially encoded IHF and FIS (factor for inversion stimulation) are participants in excisive recombination between the prophage attL and attR sites. Only Int and IHF are required for integrative recombination between the phage and bacterial at sites (attP and attB). IHF can induce sharp DNA bends in various physiological situations that may or may not involve the binding of other proteins. Similarly, HU, a nonspecific DNA binding protein closely related to IHF, has been implicated (as a multimeric array) in conferring conformational DNA changes that promote specific protein-DNA interactions. In some aspects, an engineered or recombinant IHF can be used in increasing inclusion of short fragments in a loop formation, and generating a circular product without amplification, such as, in a configuration wherein each end of the short cfNA fragment binds to one of the two random sequence overhang region of the synthetic construct.

In some aspects, the amplification can be optimized to ensure there is enough material, without amplifying unwanted materials. One of skill in the art can optimize based on various factors, including length of the DNA, nature of the template and quantities of the primer and template. In some aspects, the low cycle PCR is less than about 30 cycles, or less than about 25 cycles or less than about 20 cycles of PCR, or less than about 19, 18, 17, 16, or 15 cycles of PCR. In some aspects, a single product is obtained without amplification. In some aspects, the rolling circle amplification/extension is carried out for about 20 min-about 120 min. In some aspects, the rolling circle extension is carried on for less than 120 minutes, less than 100 minutes, less than 80 minutes, less than 60 minutes, less than 50 minutes, less than 40 minutes or less than 30 minutes. In some aspects, the rolling circle extension is performed for about 20 minutes. In some aspects, the amplification/extension is optimized to obtain about 10 fold amplification of the circularized product. In some aspects, the amplification/extension is optimized to obtain about 20 fold amplification of the circularized product. In some aspects, the amplification/extension is optimized to obtain about 30 fold amplification of the circularized product. In some aspects, the amplification/extension is optimized to obtain about 40 fold amplification of the circularized product. In some aspects, the amplification/extension is optimized to obtain about 50 fold amplification of the circularized product. In some aspects, the amplification/extension is optimized to obtain about 60 fold amplification of the circularized product. In some aspects, the amplification/extension is optimized to obtain about 70 fold amplification of the circularized product. In some aspects, the amplification/extension is optimized to obtain about 80 fold amplification of the circularized product. In some aspects, the amplification/extension is optimized to obtain about 90 fold amplification of the circularized product. In some aspects, the amplification/extension is optimized to obtain about 100 fold amplification of the circularized product.

In some aspects, rolling circle amplification reactions can be performed using ϕ-29 or Bst 2.0 DNA polymerases.

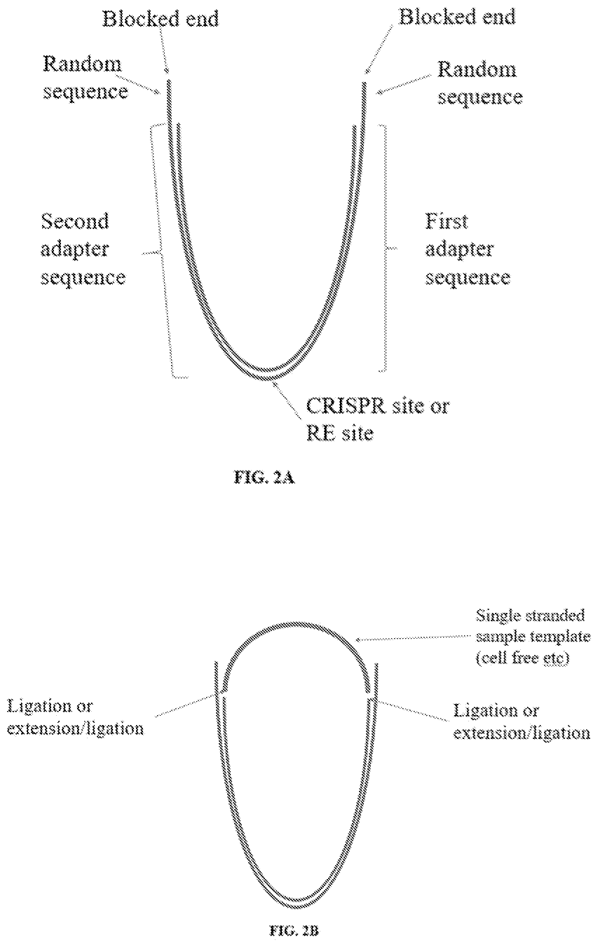

In some exemplary situations, a circularized product can be generated without the necessity of an amplification step. FIGS. 2A and 2B exemplify such aspects. In this case, a denatured cfNA strand or fragment sequence comprising a 5′ end and a 3′ end may hybridize at either end of the random sequence of a synthetic construct. The sequence of the resultant loop comprising the single stranded region of the cfNA is duplicated by extension and/or by ligation to the ends of the short stubby sequences, thereby generating the circularized product comprising the cfNA sequence. A specific sequence within the short stubby adaptors can be used to cleave the circularized product and linearize it for further amplification or assay.

In some aspects, the adapters comprise sequences representing universal handles. Sequences that are in the terminal/flanking regions of the adapters for universal applications such as identification, capture or amplification. In some aspects, the adapters comprise sequences for use in direct flow cell binding. In some aspects the adapters comprise sequences representing unique dual indexes (UDIs) 8 base unique sequences that minimize read misassignment. In some aspects the adapters comprise sequences representing 9 base unique molecular identifiers (UMIs) that can be used for quantitative assays or low-frequency variant detection. The adapters used herein can comprise unmethylated and methylated residues. Methylated residues can be processed in bisulfite PCR and sequencing, e.g. pyrosequencing.

In some aspects, adapter sequences comprise LNA or PNA for increasing stability.

### Methods Related to Amplification, Depletion and Selective Identification

Methods and uses of the compositions disclosed herein allow one to determine the sequence at any targeted site in a genome or other nucleic acid sample, including repetitive elements as well as average complexity DNA sequences, for example mRNA coding sequences. Accordingly, methods herein can be applied to any desired location in the genome, or to other repetitive or non-repetitive nucleic acid samples.

Methods of determining a nucleic acid sequence can include one or more steps of contacting a nucleic acid in a sample to an endonuclease to cleave a target nucleic acid; ligating the target nucleic acid sequence to form a circular target nucleic acid; hybridizing at least one primer to the circular target molecule to form amplified nucleic acid through rolling circle amplification; and performing sequence analysis of the amplified nucleic acid.

At least one advantage of using the techniques described in FIGS. 1A and 1B or FIGS. 2A and 2B, and detailed above, is that both DNA and RNA can be amplified. In effect, either DNA or RNA library can be prepared using the method. In effect, a library comprising any nucleic acid, DNA or RNA can be prepared using this method.

### Circularization

Circularization of target nucleic acids can utilize a ligase that enzymatically joins the 5′ end and the 3′ end of the target nucleic acid that has been cleaved by an endonuclease. In some cases, the 5′ end of the target nucleic acid is ligated directly to its 3′ end. Alternatively, the 5′ end of the target nucleic acid is joined to its 3′ end using an adapter, such as a bridge adapter that hybridizes to the 5′ end and the 3′ end of the target nucleic acid.

Any suitable ligase is contemplated to be used to circularize target nucleic acids in methods herein. Exemplary ligases include but are not limited to T7 DNA ligase, T4 DNA ligase, E. coli DNA ligase, CircLigase, T4 RNA ligase 1, T4 RNA ligase 2, Taq DNA ligase. Electroligase, SplintR ligase, or combinations thereof.

In some cases, an exonuclease is used to digest linear non-target nucleic acids that have not been circularized by earlier steps in the method. For example, the exonuclease may digest linear non-target nucleic acids from a 5′ end to a 3′ end. Alternatively, or in combination, the exonuclease may digest linear non-target nucleic acids from a 3′ end to a 5′ end. Exemplary exonucleases include but are not limited to exonuclease T, exonuclease I, thermolabile exonuclease 1, exonuclease III, exonuclease VII, exonuclease VIII, lambda exonuclease, T7 exonuclease, or combinations thereof.

In some cases the endonuclease used is a restriction endonuclease that cleaves one strand, e.g., Eco R1. In some cases the exonuclease is a λ exonuclease. In some cases, to remove the linear DNA contaminant from the nicked-circular DNA preparation, λ exonuclease treatment is used.

### Primer for Amplification

The amplification step can involve one or more primers. The primer can be selected from random primer, locus specific primer, or combinations thereof.

In some aspects, two or more primers are used in the amplification step, and the primer can include a first primer, wherein a first primer comprises at least one sequence complementary to at least a portion of the 5′ end and the 3′ end of the target nucleic acid. In some aspects, two or more primers are used in the amplification step, and the primer can include a first primer and a second primer, wherein a first primer comprises at least one sequence complementary to at least a portion of the 5′ end and the 3′ end of the target nucleic acid, and wherein a second primer comprises a sequence that is complementary to a portion of the target nucleic acid that is not adjacent to the 5′ end or the 3′ end. In some aspects, the primer contains a sequence of a region of the universal sequence.

The primer can bind to a primer recognition region on the circular nucleic acid. The primer binding sequence can be in the region of the target nucleic acid that is cut by the endonuclease and ligated by the ligase. In some aspects, the recognition site for the first nicking endonuclease is proximal to the primer binding sequence. In some aspects, the first primer binding sequence is in the region of the target nucleic acid that is cut by the endonuclease and ligated by the ligase. In one class of aspects, the recognition site for the first nicking endonuclease is proximal to the first primer binding sequence. In some cases, the primer comprises a barcode or an adapter sequence.

### Amplification and Sequencing

The target nucleic acid can be amplified using any suitable rolling circle amplification method. After a primer is annealed to the circular target nucleic acid, a strand displacing polymerase is used to extend the primer, creating multiple copies of the target nucleic acid. Strand displacing polymerases contemplated for methods herein include, but are not limited to, phi29 polymerase, Bst DNA polymerase, or combinations thereof.

The sequencing of the amplified nucleic acid can be performed concurrently with the step of rolling circle amplification. The sequencing step and the amplification step can both be performed until consensus accuracy (e.g., with a template) is reached.

The nucleic acid amplified according to the method provided herein can be sequenced according to any suitable sequencing methodology, such as direct sequencing, including sequencing by synthesis, sequencing by ligation, sequencing by hybridization, nanopore sequencing and the like. In some aspects, the immobilized DNA fragments are sequenced on a solid support. In some aspects, the solid support for sequencing is the same solid support upon which the amplification occurs. In some aspects, the sequencing is performed using a nanopore based analysis method.

Nanopore-based analysis methods often involve passing a polymeric molecule, for example single-strand DNA (“ssDNA”), through a nanoscopic opening while monitoring a signal such as an electrical signal. Typically, the nanopore is designed to have a size that allows the polymer to pass only in a sequential, single file order. As the polymer molecule passes through the nanopore, differences in the chemical and physical properties of the monomeric units that make up the polymer, for example, the nucleotides that compose the ssDNA, are translated into characteristic electrical signals.

The signal can, for example, be detected as a modulation of the ionic current by the passage of a DNA molecule through the nanopore, which current is created by an applied voltage across the nanopore-bearing membrane or film. Because of structural differences between different nucleotides, different types of nucleotides interrupt the current in different ways, with each different type of nucleotide within the ssDNA producing a type-specific modulation in the current as it passes through a nanopore, and thus allowing the sequence of the DNA to be determined.

Nanopores that have been used for sequencing DNA include protein nanopores held within lipid bilayer membranes, such as a-hemolysin nanopores, and solid state nanopores formed, for example, by ion beam sculpting of a solid-state thin film. Devices using nanopores to sequence DNA and RNA molecules have generally not been capable of reading sequence at a single-nucleotide resolution.

The step of sequencing the amplified nucleic acid can include a) providing a device comprising a substrate having an array of nanopores; each nanopore fluidically connected to an upper fluidic region and a lower fluidic region; wherein each upper fluidic region is fluidically connected through a an upper resistive opening to an upper liquid volume; and each lower fluidic region is connected to a lower liquid volume, and wherein the upper liquid volume and the lower liquid volume are each fluidically connected to two or more fluidic regions, wherein the device comprises an upper drive electrode in the upper liquid volume, a lower drive electrode in the lower liquid volume, and a measurement electrode in either the upper liquid volume or the lower liquid volume; b) placing a polymer molecule to be sequenced into one or more upper fluidic regions; c) applying a voltage across the upper and lower drive electrodes so as to pass a current through the nanopore such that the polymer molecule is translated through the nanopore; d) measuring the current through the nanopore over time; and e) using the measured current over time in step (d) to determine sequence information about the polymer molecule.

Additional sequencing methods include, but are not limited to, massively parallel signature sequencing, polony sequencing, 454 pyrosequencing, Illumina sequencing, combinatorial probe anchor synthesis, SOLiD sequencing, Ion Torrent semiconductor sequencing, DNA nanoball sequencing, Heliscope single molecule sequencing, single molecule real time sequencing, microfluidic sequencing, tunneling currents DNA sequencing, sequencing by hybridization, sequencing with mass spectrometry, RNAP sequencing, and combinations thereof.

Methods described herein can include performing a genetic analysis of the target nucleic acid. Genome sequence databases can be searched to find sequences which are related to the second nucleic acid. The search can generally be performed by using computer-implemented search algorithms to compare the query sequences with sequence information stored in a plurality of databases accessible via a communication network, for example, the Internet. Examples of such algorithms include the Basic Local Alignment Search Tool (BLAST) algorithm, the PSI-blast algorithm, the Smith-Waterman algorithm, the Hidden Markov Model (HMM) algorithm, and other like algorithms.

A number of sequence-specific cleavage approaches can be used to deplete target nucleic acids so as to enrich for nucleic acid of interest. These techniques, including Zinc Finger Nucleases (ZFN), Transcription activator like effector nucleases and Clustered Regulatory Interspaced Short Palindromic Repeat/Cas based RNA guided DNA nuclease (CRISPR/Cas9) allow for sequence specific degradation of double stranded DNA. Alternately, restriction endonuclease, particularly restriction endonucleases that have cleavage specificity that targets particular regions to be depleted while preferably leaving other nucleic acid molecules uncleaved, are also compatible with the disclosure herein. In some aspects, a repeat-region specific endonuclease such as an Alu restriction endonuclease or other transposon or repeat region specific endonuclease is selected so as to deplete the corresponding nucleic acids from a sample. These techniques can be used to, for example, cleave the first nucleic acid at one or more sites to generate an exposed end or set of exposed ends available for exonuclease degradation. The ability to target sequence specific locations for double stranded DNA cuts makes these genome editing tools compatible with depletion of a redundant or otherwise undesired target nucleic acid in the sample.

A sample subjected to selective depletion comprises sequence of the first nucleic acid and the second nucleic acid. In some aspects a target sample comprises non-repetitive sequence and repetitive sequence. In some aspects a target sample comprises single-copy sequence and multi-copy sequence. In some cases a host sample is fragmented and differentially degraded so as, for example, to selectively remove repetitive regions of a genome while leaving high-information regions undegraded and therefore selectively enriched. In some aspects, a sample comprises blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free DNA, such as tumor DNA or fetal DNA.

Provided herein are methods, compositions and kits related to the selective enrichment of nucleic acids of interest, such as selective enrichment of pathogen nucleic acids, symbiote nucleic acids, microbiome nucleic acids, high information regions, cancer alleles, or other nucleic acids of interest in a sample.

In some cases, the first nucleic acid is from a host. In some cases, the first nucleic acid is from one or more hosts selected from the group consisting of mammals, such as a human, cow, horse, sheep, pig, monkey, dog, cat, gerbil, bird, mouse, and rat, or any mammalian laboratory model for a disease, condition or other phenomenon involving rare nucleic acids. In some cases, the first nucleic acid is from a human. Some of examples of the second nucleic acid, e.g., the nucleic acid of interest can be from pathogens, microbiomes, tumor, fetal DNA in a maternal sample, alleles, and mutant alleles. In some cases, the second nucleic acid is from a non-host. In some cases, the second nucleic acid is from a prokaryotic organism. In some cases, the second nucleic acid is from one or more selected from the group consisting of a eukaryote, virus, bacterial, fungus, and protozoa. In some aspects, the second nucleic acid can be from tumor cells. In some aspects, the second nucleic acid can be fetal DNA in a maternal sample. In some aspects, the second nucleic acid can be alleles or mutant alleles. Microbiomes are also sources of second nucleic acids consistent with the disclosure herein, as are other examples apparent to one of skill in the art.

In some cases, the first nucleic acid and the second nucleic acid are capped at the 5′ and 3′ ends in order to protect the ends from exonuclease digestion. In some aspects, the first nucleic acid and the second nucleic acid are capped by attaching an adapter. In some aspects, attaching comprises ligating. In some aspects, the first nucleic acid and the second nucleic acid are capped by a chemical modification to the 5′ and the 3′ ends. In some aspects, the cap comprises a phosphorthioate. In some aspects, the cap comprises a 2′ modified nucleoside, such as a 2′-O-modified ribose, a 2′-O-methyl nucleoside, or a 2′-O-methoxyethyl nucleoside. In some aspects, the cap comprises an inverted dT modification. Additional methods of capping and protecting the ends of nucleic acids are provided elsewhere herein.

In some cases, the first nucleic acid capped with an adapter having a size in a range from about 10 bp to about 50 bp.

In some aspects, depletion of a host nucleic acid is performed to enrich detection of a pathogenic nucleic acid signature. In some aspects a depletion step results in about 80%, 81%, 82%, 83%, 84%, 85%, 86%, 87%, 88%, 89%, 90%, 91%, 92%, 93%, 94%, 95%, 96%, 97%, 98%, 99% or 100% depletion of the undesired nucleic acid (e.g., host nucleic acid). In some aspects, depletion of a pathogenic or contaminant nucleic acid is performed to enrich detection of a host (e.g., human) nucleic acid signature. In some aspects a depletion step results in about 80%, 81%, 82%, 83%, 84%, 85%, 86%, 87%, 88%, 89%, 90%, 91%, 92%, 93%, 94%, 95%, 96%, 97%, 98%, 99% or 100% depletion of the undesired nucleic acid (e.g., pathogenic nucleic acid).

In some aspects a depletion step results in about 80%, 81%, 82%, 83%, 84%, 85%, 86%, 87%, 88%, 89%, 90%, 91%, 92%, 93%, 94%, 95%, 96%, 97%, 98%, 99% or 100% depletion of the undesired nucleic acid in a sample (e.g., ribosomal nucleic acid).

In some aspects a depletion step results in about 80%, 81%, 820/%, 83%, 84%, 85%, 86%, 87%, 88%, 89%, 90%, 91%, 92%, 93%, 94%, 95%, 96%, 97%, 98%, 99% or 100% depletion of the undesired nucleic acid (e.g., host nucleic acid).

In some aspects, a first nucleic acid is a desired nucleic acid, is selectively enriched in a sample comprising heterogenous composition, by depleting a second nucleic acid also present in the composition, which is the undesired nucleic acid or contaminant. In some aspects, depletion is performed by site specific endonucleases such as DNA guided endonuclease, for example Argonaute (AGO).

In some aspects a moiety that specifically binds to the first nucleic acid comprises a guide RNA molecule. In some aspects a population of moieties that specifically bind to first nucleic acid comprises a population of guide RNA molecules, such as a population of guide molecules that bind to the first nucleic acid.

### Endonuclease for Targeted Cleavage of Nucleic Acid

Methods disclosed herein comprise targeting cleavage of the first nucleic acid using a site-specific, targetable, and/or engineered nuclease or nuclease system. Such nucleases may create double-stranded break (DSBs) at desired locations in a genomic, cDNA or other nucleic acid molecule. In other examples, a nuclease may create a single strand break. In some cases, two nucleases are used, each of which generates a single strand break. Many cleavage enzymes consistent with the disclosure herein share a trait that they yield molecules having an end accessible for single stranded or double stranded exonuclease activity.

The endonuclease used herein can be a restriction enzyme specific to at least one site on the first nucleic acid and that does not cleave a second nucleic acid. The endonuclease described herein can be specific to a repetitive nucleic sequence in a host genome, such as a transposon or other repeat, a centromeric region, or other repeat sequence. For example, some restriction endonucleases consistent with the disclosure herein are Alu specific restriction enzymes. A restriction is Alu specific or, for that matter, other target ‘specific’ if it cuts a target and does not cut other substrates, or cuts other targets infrequently so as to differentially deplete its ‘specific’ target. The presence of a non-Alu or other non-target cleavage, such as due to the rare occurrence of the cleavage site elsewhere in a host genome or transcriptome, or in a pathogen or other rare nucleic acid present in a sample, does not render an endonuclease ‘nonspecific’ so long as differential depletion of undesired nucleic acid is effected.

The first nucleic acid can include a restriction enzyme Alu recognition site. The second nucleic acid does not include the Alu recognition site. In some aspects, the first nucleic acid comprises at least one sequence that maps to at least one nucleic acid recognition site selected from the group consisting of recognition sites of AluI, AsuHPI, Bpu10I, BssECI, BstDEI, BstMAI, HinfI, and BstTUI. In some aspects, the second nucleic acid does not include at least one of the recognition sites selected from recognition sites of AluI, AsuHPI, Bpu10I, BssECI, BstDEI, BstMAI, HinfI, and BstTUI.

Endonucleases consistent with the disclosure herein variously include at least one selected from Clustered Regulatory Interspaced Short Palindromic Repeat (CRISPR)/Cas system protein-gRNA complexes, Zinc Finger Nucleases (ZFN), and Transcription activator like effector nucleases. In some aspects, the gRNAs are complementary to at least one site on the first nucleic acid to generate cleaved first nucleic acids capped only on one end. Other programmable, nucleic acid sequence specific endonucleases are also consistent with the disclosure herein.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems can be used, such as CRISPR/Cas systems including c2c2 nucleases.

Methods disclosed herein may comprise cleaving a target nucleic acid using CRISPR systems, such as a Type I, Type II, Type III, Type IV, Type V, or Type VI CRISPR system. CRISPR/Cas systems can be multi-protein systems or single effector protein systems. Multi-protein, or Class 1, CRISPR systems include Type I, Type III, and Type IV systems. Alternatively, Class 2 systems include a single effector molecule and include Type II, Type V, and Type VI.

CRISPR systems used in some methods disclosed herein may comprise a single or multiple effector proteins. An effector protein may comprise one or multiple nuclease domains. An effector protein may target DNA or RNA, and the DNA or RNA can be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS can be 3′ or 5′ of the target or protospacer site. Cleavage of a target sequence may generate blunt ends, 3′ overhangs, or 5′ overhangs. In some cases, target nucleic acids do not comprise a PAM or PFS.

A gRNA may comprise a spacer sequence. In some aspects, spacer sequences are complementary to at least a portion of target sequences or protospacer sequences. Spacer sequences can be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence can be less than 10 or more than 36 nucleotides in length.

In some aspects, a gRNA comprises a repeat sequence. In some cases, the repeat sequence is part of a double stranded portion of the gRNA. A repeat sequence can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence can be less than 10 or more than 50 nucleotides in length.

In some aspects, a gRNA comprises one or more synthetic nucleotides, non-naturally occurring nucleotides, nucleotides with a modification, deoxyribonucleotide, or any combination thereof. Additionally or alternatively, a gRNA comprises a hairpin, linker region, single stranded region, double stranded region, or any combination thereof. Additionally or alternatively, a gRNA may comprise a signaling or reporter molecule.

A CRISPR nuclease can be endogenously or recombinantly expressed. A CRISPR nuclease can be encoded on a chromosome, extrachromosomally, or on a plasmid, synthetic chromosome, or artificial chromosome. A CRISPR nuclease can be provided as a polypeptide or mRNA encoding the polypeptide. In such examples, polypeptide or mRNA can be delivered through standard mechanisms known in the art, such as through the use of cell permeable peptides, nanoparticles, or viral particles.

In some aspects, gRNAs are encoded by genetic or episomal DNA. gRNAs can be provided or delivered concomitantly with a CRISPR nuclease or sequentially. Guide RNAs can be chemically synthesized, in vitro transcribed or otherwise generated using standard RNA generation techniques known in the art.

In some aspects, a CRISPR system is a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM can be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which is some cases creates two blunt ends. In some cases, the Type II CRISPR nuclease is engineered to be a nickase such that the nuclease only generates a single strand break. In such cases, two distinct nucleic acid sequences can be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease can be catalytically dead such that it binds to a target sequence, but does not cleave. For example, a Type II nuclease may have mutations in both the RuvC and HNH domains, thereby rendering both nuclease domains non-functional. A Type II CRISPR system can be one of three sub-types, namely Type II-A, Type II-B, or Type II-C.

In some aspects, a CRISPR system is a Type V CRISPR system, for example a Cpf1, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as can be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences can be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease can be catalytically dead such that it binds to a target sequence, but does not cleave. For example, a Type V nuclease could have mutations a RuvC domain, thereby rendering the nuclease domain non-functional.

In some aspects, a CRISPR system is a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS can be 3′ or 5′ or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease can be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems can be used to cleave certain nucleic acid sequences. Ago protein can be derived from a prokaryote, eukaryote, or archaea. The nucleic acid contemplated can be RNA or DNA. A DNA target can be single stranded or double stranded. In some examples, the certain nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins can be used in combination to generate a double strand break. In some examples, an Ago protein comprises one, two, or more nuclease domains. In some examples, an Ago protein comprises one, two, or more catalytic domains. One or more nuclease or catalytic domains can be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins can be targeted to target nucleic acid sequences by a guiding nucleic acid. In many examples, the guiding nucleic acid is a guide DNA (gDNA). The gDNA can have a 5′ phosphorylated end. The gDNA can be single stranded or double stranded. Single stranded gDNA can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA can be less than 10 nucleotides in length. In some examples, the gDNA can be more than 50 nucleotides in length.

Argonaute-mediated cleavage can generate blunt end, 5′ overhangs, or 3′ overhangs. In some examples, one or more nucleotides are removed from the target site during or following cleavage.

Argonaute protein can be endogenously or recombinantly expressed. Argonaute can be encoded on a chromosome, extrachromosomally, or on a plasmid, synthetic chromosome, or artificial chromosome. Additionally or alternatively, an Argonaute protein can be provided as a polypeptide or mRNA encoding the polypeptide. In such examples, polypeptide or mRNA can be delivered through standard mechanisms known in the art, such as through the use of peptides, nanoparticles, or viral particles.

Guide DNAs can be provided by genetic or episomal DNA. In some examples, gDNA are reverse transcribed from RNA or mRNA. In some examples, guide DNAs can be provided or delivered concomitantly with an Ago protein or sequentially. Guide DNAs can be chemically synthesized, assembled, or otherwise generated using standard DNA generation techniques known in the art. Guide DNAs can be cleaved, released, or otherwise derived from genomic DNA, episomal DNA molecules, isolated nucleic acid molecules, or any other source of nucleic acid molecules.

Nuclease fusion proteins can be recombinantly expressed. A nuclease fusion protein can be encoded on a chromosome, extrachromosomally, or on a plasmid, synthetic chromosome, or artificial chromosome. A nuclease and a chromatin-remodeling enzyme can be engineered separately, and then covalently linked. A nuclease fusion protein can be provided as a polypeptide or mRNA encoding the polypeptide. In such examples, polypeptide or mRNA can be delivered through standard mechanisms known in the art, such as through the use of peptides, nanoparticles, or viral particles.

A guide nucleic acid may complex with a compatible nucleic acid-guided nuclease and may hybridize with a target sequence, thereby directing the nuclease to the target sequence. A subject nucleic acid-guided nuclease capable of complexing with a guide nucleic acid can be referred to as a nucleic acid-guided nuclease that is compatible with the guide nucleic acid. Likewise, a guide nucleic acid capable of complexing with a nucleic acid-guided nuclease can be referred to as a guide nucleic acid that is compatible with the nucleic acid-guided nucleases.

A guide nucleic acid can be DNA. A guide nucleic acid can be RNA. A guide nucleic acid may comprise both DNA and RNA. A guide nucleic acid may comprise modified of non-naturally occurring nucleotides. In cases where the guide nucleic acid comprises RNA, the RNA guide nucleic acid can be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment can be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence can be 10-25 nucleotides in length. The guide sequence can be 10-20 nucleotides in length. The guide sequence can be 15-30 nucleotides in length. The guide sequence can be 20-30 nucleotides in length. The guide sequence can be 15-25 nucleotides in length. The guide sequence can be 15-20 nucleotides in length. The guide sequence can be 20-25 nucleotides in length. The guide sequence can be 22-25 nucleotides in length. The guide sequence can be 15 nucleotides in length. The guide sequence can be 16 nucleotides in length. The guide sequence can be 17 nucleotides in length. The guide sequence can be 18 nucleotides in length. The guide sequence can be 19 nucleotides in length. The guide sequence can be 20 nucleotides in length. The guide sequence can be 21 nucleotides in length. The guide sequence can be 22 nucleotides in length. The guide sequence can be 23 nucleotides in length. The guide sequence can be 24 nucleotides in length. The guide sequence can be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment can be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions can be 10-25 nucleotides in length. At least one of the two sequence regions can be 10-20 nucleotides in length. At least one of the two sequence regions can be 15-30 nucleotides in length. At least one of the two sequence regions can be 20-30 nucleotides in length. At least one of the two sequence regions can be 15-25 nucleotides in length. At least one of the two sequence regions can be 15-20 nucleotides in length. At least one of the two sequence regions can be 20-25 nucleotides in length. At least one of the two sequence regions can be 22-25 nucleotides in length. At least one of the two sequence regions can be 15 nucleotides in length. At least one of the two sequence regions can be 16 nucleotides in length. At least one of the two sequence regions can be 17 nucleotides in length. At least one of the two sequence regions can be 18 nucleotides in length. At least one of the two sequence regions can be 19 nucleotides in length. At least one of the two sequence regions can be 20 nucleotides in length. At least one of the two sequence regions can be 21 nucleotides in length. At least one of the two sequence regions can be 22 nucleotides in length. At least one of the two sequence regions can be 23 nucleotides in length. At least one of the two sequence regions can be 24 nucleotides in length. At least one of the two sequence regions can be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In some aspects of the disclosure, the term “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid can be compatible with a nucleic acid-guided nuclease when the two elements may form a functional targetable nuclease complex capable of cleaving a target sequence. Often, a compatible scaffold sequence for a compatible guide nucleic acid can be found by scanning sequences adjacent to native nucleic acid-guided nuclease loci. In other words, native nucleic acid-guided nucleases can be encoded on a genome within proximity to a corresponding compatible guide nucleic acid or scaffold sequence.

Nucleic acid-guided nucleases can be compatible with guide nucleic acids that are not found within the nucleases endogenous host. Such orthogonal guide nucleic acids can be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid can be engineered to target a desired target sequence by altering the guide sequence such that the guide sequence is complementary to the target sequence, thereby allowing hybridization between the guide sequence and the target sequence. A guide nucleic acid with an engineered guide sequence can be referred to as an engineered guide nucleic acid. Engineered guide nucleic acids are often non-naturally occurring and are not found in nature.

In some aspects the guide RNA molecule interferes with sequencing directly, for example by binding the target sequence to prevent nucleic acid polymerization to occur across the bound sequence. In some aspects the guide RNA molecule works in tandem with a RNA-DNA hybrid binding moiety such as a protein. In some aspects the guide RNA molecule directs modification of member of the sequencing library to which it may bind, such as methylation, base excision, or cleavage, such that in some aspects the member of the sequencing library to which it is bound becomes unsuitable for further sequencing reactions. In some aspects, the guide RNA molecule directs endonucleolytic cleavage of the DNA molecule to which it is bound, for example by a protein having endonuclease activity such as Cas9 protein. Zinc Finger Nucleases (ZFN), Transcription activator like effector nucleases and Clustered Regulatory Interspaced Short Palindromic Repeat/Cas based RNA guided DNA nuclease (CRISPR/Cas9), among others, are compatible with some aspects of the disclosure herein.

A guide RNA molecule comprises sequence that base-pairs with target sequence that is to be removed from sequencing (the first nucleic acid). In some aspects the base-pairing is complete, while in some aspects the base pairing is partial or comprises bases that are unpaired along with bases that are paired to non-target sequence.

A guide RNA may comprise a region or regions that form an RNA ‘hairpin’ structure. Such region or regions comprise partially or completely palindromic sequence, such that 5′ and 3′ ends of the region may hybridize to one another to form a double-strand ‘stem’ structure, which in some aspects is capped by a non-palindromic loop tethering each of the single strands in the double strand loop to one another.

In some aspects, the guide RNA comprises a stem loop such as a tracrRNA stem loop. A stem loop such as a tracrRNA stem loop may complex with or bind to a nucleic acid endonuclease such as Cas9 DNA endonuclease. Alternately, a stem loop may complex with an endonuclease other than Cas9 or with a nucleic acid modifying enzyme other than an endonuclease, such as a base excision enzyme, a methyltransferase, or an enzyme having other nucleic acid modifying activity that interferes with one or more DNA polymerase enzymes.

The tracrRNA/CRISPR/Endonuclease system was identified as an adaptive immune system in eubacterial and archaeal prokaryotes whereby cells gain resistance to repeated infection by a virus of a known sequence. See, for example, Deltcheva E, Chylinski K, Sharma C M, Gonzales K, Chao Y, Pirzada Z A et al. (2011) “CRISPR RNA maturation by trans-encoded small RNA and host factor RNase III” Nature 471 (7340): 602-7. doi:10.1038/nature09886. PMC 3070239. PMID 21455174; Terns M P, Terns R M (2011) “CRISPR-based adaptive immune systems” Curr Opin Microbiol 14 (3): 321-7. doi:10.1016/j.mib.2011.03.005. PMC 3119747. PMID 21531607; Jinek M, Chylinski K, Fonfara I, Hauer M, Doudna J A, Charpentier E (2012) “A Programmable Dual-RNA-Guided DNA Endonuclease in Adaptive Bacterial Immunity” Science 337 (6096): 816-21. doi:10.1126/science.1225829. PMID 22745249; and Brouns S J (2012) “A swiss army knife of immunity” Science 337 (6096): 808-9. doi:10.1126/science.1227253. PMID 22904002. The system has been adapted to direct targeted mutagenesis in eukaryotic cells. See, e.g., Wenzhi Jiang, Huanbin Zhou, Honghao Bi, Michael Fromm, Bing Yang, and Donald P. Weeks (2013) “Demonstration of CRISPR/Cas9/sgRNA-mediated targeted gene modification in Arabidopsis, tobacco, sorghum and rice” Nucleic Acids Res. November 2013; 41(20): e188, Published online Aug. 31, 2013. doi: 10.1093/nar/gkt780, and references therein.

As contemplated herein, a guide RNA is used in some aspects to provide sequence specificity to a DNA endonuclease such as a Cas9 endonuclease. In these aspects, a guide RNA comprises a hairpin structure that binds to or is bound by an endonuclease such as Cas9 (other endonucleases are contemplated as alternatives or additions in some aspects), and a guide RNA further comprises a recognition sequence that binds to or specifically binds to or exclusively binds to a sequence that is to be removed from a sequencing library or a sequencing reaction. The length of the recognition sequence in a guide RNA may vary according to the degree of specificity desired in the sequence elimination process. Short recognition sequences, comprising frequently occurring sequence in the sample or comprising differentially abundant sequence (abundance of AT in an AT-rich genome sample or abundance of GC in a GC-rich genome sample) are likely to identify a relatively large number of sites and therefore to direct frequent nucleic acid modification such as endonuclease activity, base excision, methylation or other activity that interferes with at least one DNA polymerase activity. Long recognition sequences, comprising infrequently occurring sequence in the sample or comprising underrepresented base combinations (abundance of GC in an AT-rich genome sample or abundance of AT in a GC-rich genome sample) are likely to identify a relatively small number of sites and therefore to direct infrequent nucleic acid modification such as endonuclease activity, base excision, methylation or other activity that interferes with at least one DNA polymerase activity. Accordingly, as disclosed herein, in some aspects one may regulate the frequency of sequence removal from a sequence reaction through modifications to the length or content of the recognition sequence.

Guide RNA can be synthesized through a number of methods consistent with the disclosure herein. Standard synthesis techniques can be used to produce massive quantities of guide RNAs, and/or for highly-repetitive targeted regions, which may require only a few guide RNA molecules to target a multitude of unwanted loci. The double stranded DNA molecules can comprise an RNA site specific binding sequence, a guide RNA sequence for Cas9 protein and a T7 promoter site. In some cases, the double stranded DNA molecules can be less than about 100 bp length. T7 polymerase can be used to create the single stranded RNA molecules, which may include the target RNA sequence and the guide RNA sequence for the Cas9 protein.

Guide RNA sequences can be designed through a number of methods. For example, in some aspects, non-genic repeat sequences of the human genome are broken up into, for example, 100 bp sliding windows. Double stranded DNA molecules can be synthesized in parallel on a microarray using photolithography.

The target sequence windows may vary in size. 30-mer target sequences can be designed with a short trinucleotide protospacer adjacent motif (PAM) sequence of N-G-G flanking the 5′ end of the target design sequence, which in some cases facilitates cleavage. &e, among others, Giedrius Gasiunas et al., (2012) “Cas9-crRNA ribonucleoprotein complex mediates specific DNA cleavage for adaptive immunity in bacteria” Proc. Natl. Acad. Sci. USA. September 25, 109(39): E2579-E2586, which is hereby incorporated by reference in its entirety. Redundant sequences can be eliminated and the remaining sequences can be analyzed using a search engine (e.g. BLAST) against the human genome to avoid hybridization against REFSEQ, ENSEMBL and other gene databases to avoid nuclease activity at these sites. The universal Cas9 tracer RNA sequence can be added to the guide RNA target sequence and then flanked by the T7 promoter. The sequences upstream of the T7 promoter site can be synthesized. Due to the highly repetitive nature of the target regions in the human genome, in many aspects, a relatively small number of guide RNA molecules will digest a larger percentage of NGS library molecules.

Although only about 50% of protein coding genes are estimated to have exons comprising the NGG PAM (protospacer adjacent motif) sequence, multiple strategies are provided herein to increase the percentage of the genome that can be targeted with the Cas9 cutting system. For example, if a PAM sequence is not available in a DNA region, a PAM sequence can be introduced via a combination strategy using a guide RNA coupled with a helper DNA comprising the PAM sequence. The helper DNA can be synthetic and/or single stranded. The PAM sequence in the helper DNA will not be complimentary to the gDNA knockout target in the NGS library, and may therefore be unbound to the target NGS library template, but it can be bound to the guide RNA. The guide RNA can be designed to hybridize to both the target sequence and the helper DNA comprising the PAM sequence to form a hybrid DNA:RNA:DNA complex that can be recognized by the Cas9 system.

The PAM sequence can be represented as a single stranded overhang or a hairpin. The hairpin can, in some cases, comprise modified nucleotides that may optionally be degraded. For example, the hairpin can comprise Uracil, which can be degraded by Uracil DNA Glycosylase.

As an alternative to using a DNA comprising a PAM sequence, modified Cas9 proteins without the need of a PAM sequence or modified Cas9 with lower sensitivity to PAM sequences can be used without the need for a helper DNA sequence.

In further cases, the guide RNA sequence used for Cas9 recognition can be lengthened and inverted at one end to act as a dual cutting system for close cutting at multiple sites. The guide RNA sequence can produce two cuts on a NGS DNA library target. This can be achieved by designing a single guide RNA to alternate strands within a restricted distance. One end of the guide RNA may bind to the forward strand of a double stranded DNA library and the other may bind to the reverse strand. Each end of the guide RNA can comprise the PAM sequence and a Cas9 binding domain. This may result in a dual double stranded cut of the NGS library molecules from the same DNA sequence at a defined distance apart.

In some instances, the assay comprise at least one sequence-specific nuclease, and in some cases a combination of sequence-specific nucleases, such as at least one restriction endonuclease having a recognition site that is abundant in the first nucleic acid. In some cases an enzyme comprises an activity that yields double-stranded breaks in response to a specific sequence. In some cases, an enzyme comprises any nuclease or other enzyme that digests double-stranded nucleic acid material in RNA/DNA hybrids.

Nucleic acid probes (e.g. biotinylated probes) complementary to the second nucleic acids can be hybridized to the second nucleic acids in solution and pulled down with, e.g., magnetic streptavidin-coated beads. Unbound nucleic acids can be washed away and the captured nucleic acids may then be eluted and amplified for sequencing or genotyping.

In some aspects, practice of the methods herein reduces the sequencing time duration of a sequencing reaction, such that a nucleic acid library is sequenced in a shorter time, or using fewer reagents, or using less computing power. In some aspects, practice of the methods herein reduces the sequencing time duration of a sequencing reaction for a given nucleic acid library to about 90%, 80%, 70%, 600, 50%, 40%, 33%, 30% or less than 30% of the time required to sequence the library in the absence of the practice of the methods herein.

In some aspects, a specific read sequence from a specific region is of particular interest in a given sequencing reaction. Measures to allow the rapid identification of such a specific region are beneficial as they may decrease computation time or reagent requirements or both computation time and reagent requirements.

Some aspects of the disclosure relate to the generation of guide RNA molecules. Guide RNA molecules are in some cases transcribed from DNA templates. A number of RNA polymerases can be used, such as T7 polymerase, RNA PolI, RNA PolII, RNA PolIII, an organellar RNA polymerase, a viral RNA polymerase, or a eubacterial or archaeal polymerase. In some cases the polymerase is T7.

Guide RNA generating templates comprise a promoter, such as a promoter compatible with transcription directed by T7 polymerase, RNA PolI, RNA PolII, RNA PolIII, an organellar RNA polymerase, a viral RNA polymerase, or a eubacterial or archaeal polymerase. In some cases the promoter is a T7 promoter.

Guide RNA templates encode a tag sequence in some cases. A tag sequence binds to a nucleic acid modifying enzyme such as a methylase, base excision enzyme or an endonuclease. In the context of a larger Guide RNA molecule bound to a nontarget site, a tag sequence tethers an enzyme to a nucleic acid nontarget region, directing activity to the nontarget site. An exemplary tethered enzyme is an endonuclease such as Cas9.

Guide RNA templates are complementary to the first nucleic acid corresponding to ribosomal RNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

In many cases, the tag sequence comprises a stem-loop, such as a partial or total stem-loop structure. The ‘stem’ of the stem loop structure is encoded by a palindromic sequence in some cases, either complete or interrupted to introduce at least one ‘kink’ or turn in the stem. The ‘loop’ of the stem loop structure is not involved in stem base pairing in most cases. In some cases, the stem loop is encoded by a tracr sequence, such as a tracr sequence disclosed in references incorporated herein. Some stem loops bind, for example, Cas9 or other endonuclease.

Guide RNA molecules additionally comprise a recognition sequence. The recognition sequence is completely or incompletely reverse-complementary to a nontarget sequence to be eliminated from a nucleic acid library sequence set. As RNA is able to hybridize using base pair combinations (G:U base pairing, for example) that do not occur in DNA-DNA hybrids, the recognition sequence does not need to be an exact reverse complement of the nontarget sequence to bind. In addition, small perturbations from complete base pairing are tolerated in some cases.

### End Protection

Protecting the ends of DNA molecules from degradation can be effected through a number of approaches, provided that an end result is prevention of adapter-added fragments from exonuclease degradation at the site of adapter attachment. Adapters are added through ligation, polymerase mediated amplification, tagmentation via transposase delivery, end modification or other approaches. Representative adapters include hairpin adapters that effectively link the two strands of a double-stranded nucleic acid to form a single-stranded circular molecule if added at both ends. Such a molecule lacks an exposed end for single stranded or double stranded exonuclease degradation unless it is further cleaved by an endonuclease. Protection is also effected by attachment of an oligonucleotide or other molecule that is resistant to exonuclease activity. Examples of exonuclease-resistant adapters include phosphorothioate oligos, 2-O methyl modified nucleotide sugars, inverted dT or ddT, phosphorylation, C3 spacers or other modifications that inhibit an exonuclease from traversing the modification so as do degrade adjacent nucleic acids. Alternately or in combination, in some cases an ‘adapter’ constitutes modification to the ends of sample nucleic acids without ligation of additional molecules, such that the modification renders the nucleic acids resistant to exonuclease degradation.

A particular feature of the adapters herein is that, although they operate locally independent of one another, a nucleic acid is not protected from degradation unless both ends are subjected to adapter addition or modification. In some embodiments, adapter end is protected from exonuclease activity, the opposite end of the nucleic acid is vulnerable to degradation such that the molecule as a whole is degraded. This is the fate of nucleic acids that are adapter modified but then cleaved by a sequence-specific nucleic acid endonuclease as contemplated herein, so as to yield at least two exposed, unprotected nucleic acid ends. In some embodiments, the 3′ ends of adapters RS1 and RS2 are protected from ligation.

### Non-Host Nucleic Acids

Targeted depletion methods herein result in removal of a first nucleic acid and enrichment of a second nucleic acid from the sample. Said sample can be used to make a library for sequencing and said sequencing delivers sequence data that can be mostly derived from the second nucleic acid. For example, the second nucleic acid can be a non-host nucleic acid.

In certain aspects, provided herein are methods that result in enrichment of sequences originated from a microbial pathogen. In some cases, methods herein enable identification of said microbial pathogen. In some aspects the microbial pathogen comprises a bacterial pathogen. In some aspects, the bacterial pathogen is a Bacillus such as a Bacillus anthracis or a Bacillus cereus; a Bartonella such as a Bartonella henselae or a Bartonella quintana; a Bordetella such as a Bordetella pertussis; a Borrelia such as a Borrelia burgdorferi, a Borrelia garinii, a Borrelia afzelii, a Borrelia recurrentis; a Brucella such as a Brucella abortus, a Brucella canis, a Brucella melitensis or a Brucella suis; a Campylobacter such as a Campylobacter jejuni; a Chlamydia or Chlamydophila such as Chlamydia pneumoniae, Chlamydia trachomatis, Chlamydophila psittaci; a Clostridium such as a Clostridium botulinum, a Clostridium difficile, a Clostridium perfringens, a Clostridium tetani; a Corynebacterium such as a Corynebacterium diphtheriae; an Enterococcus such as a Enterococcus faecalis or a Enterococcus faecium; a Escherichia such as a Escherichia coli; a Francisella such as a Francisella tularensis; a Haemophilus such as a Haemophilus influenzae; a Helicobacter such as a Helicobacter pylori; a Legionella such as a Legionella pneumophila; a Leptospira such as a Leptospira interrogans, a Leptospira santarosai, a Leptospira weilii or a Leptospira noguchii; a Listeria such as a Listeria monocytogenes; a Mycobacterium such as a Mycobacterium leprae, a Mycobacterium tuberculosis or a Mycobacterium ulcerans; a Mycoplasma such as a Mycoplasma pneumoniae; a Neisseria such as a Neisseria gonorrhoeae or a Neisseria meningitidis; a Pseudomonas such as a Pseudomonas aeruginosa; a Rickettsia such as a Rickettsia rickettsii; a Salmonella such as a Salmonella typhi or a Salmonella typhimurium; a Shigella such as a Shigella sonnei; a Staphylococcus such as a Staphylococcus aureus, a Staphylococcus epidermidis, a Staphylococcus saprophyticus; a Streptococcus such as a Streptococcus agalactiae, a Streptococcus pneumoniae, a Streptococcus pyogenes; a Treponema such as a Treponema pallidum; a Vibrio such as a Vibrio cholerae; a Yersinia such as a Yersinia pestis, a Yersinia enterocolitica or a Yersinia pseudotuberculosis. In some aspects, the microbial pathogen comprises a viral pathogen. In some aspects, the viral pathogen comprises a Adenoviridae such as, an Adenovirus; a Herpesviridae such as a Herpes simplex, type 1, a Herpes simplex, type 2, a Varicella-zoster virus, an Epstein-barr virus, a Human cytomegalovirus, a Human herpesvirus, type 8; a Papillomaviridae such as a Human papillomavirus; a Polyomaviridae such as a BK virus or a JC virus; a Poxviridae such as a Smallpox; a Hepadnaviridae such as a Hepatitis B virus; a Parvoviridae such as a Human bocavirus or a Parvovirus; a Astroviridae such as a Human astrovirus; a Caliciviridae such as a Norwalk virus; a Picornaviridae such as a coxsackievirus, a hepatitis A virus, a poliovirus, a rhinovirus; a Coronaviridae such as a Severe acute respiratory syndrome virus or a Wuhan coronavirus; a Flaviviridae such as a Hepatitis C virus, a yellow fever virus, a dengue virus, a West Nile virus; a Togaviridae such as a Rubella virus; a Hepeviridae such as a Hepatitis E virus; a Retroviridae such as a Human immunodeficiency virus (HIV); a Orthomyxoviridae such as an Influenza virus; a Arenaviridae such as a Guanarito virus, a Junin virus, a Lassa virus, a Machupo virus, a Sabia virus; a Bunyaviridae such as a Crimean-Congo hemorrhagic fever virus; a Filoviridae such as a Ebola virus, a Marburg virus; a Paramyxoviridae such as a Measles virus, a Mumps virus, a Parainfluenza virus, a Respiratory syncytial virus, a Human metapneumovirus, a Hendra virus, a Nipah virus; a Rhabdoviridae such as a Rabies virus; a Hepatitis D virus; or a Reoviridae such as a Rotavirus, a Orbivirus, a Coltivirus, a Banna virus pathogen. In some aspects, the microbial pathogen comprises a fungal pathogen. In some aspects, the fungal pathogen comprises actinomycosis, allergic bronchopulmonary aspergillosis, aspergilloma, aspergillosis, athlete's foot, basidiobolomycosis, basidiobolus ranarum, black piedra, blastomycosis, candida krusei, candidiasis, chronic pulmonary aspergillosis, chrysosporium, chytridiomycosis, coccidioidomycosis, conidiobolomycosis, cryptococcosis, cryptococcus gattii, deep dermatophytosis, dermatophyte, dermatophytid, dermatophytosis, endothrix, entomopathogenic fungus, epizootic lymphangitis, esophageal candidiasis, exothrix, fungal meningitis, fungemia, geotrichum, geotrichum candidum, histoplasmosis, lobomycosis, massospora cicadina, microsporum gypseum, muscardine, mycosis, myringomycosis, neozygites remaudierei, neozygites slavi, ochroconis gallopava, ophiocordyceps arborescens, ophiocordyceps coenomyia, ophiocordyceps macroacicularis, ophiocordyceps nutans, oral candidiasis, paracoccidioidomycosis, pathogenic dimorphic fungi, penicilliosis, piedra, piedraia, pneumocystis pneumonia, pseudallescheriasis, scedosporiosis, sporotrichosis, tinea, tinea barbae, tinea capitis, tinea corporis, tinea cruris, tinea faciei, tinea incognito, tinea nigra, tinea pedis, tinea versicolor, vomocytosis, white nose syndrome, zeaspora, or zygomycosis. In some cases, methods herein result in enrichment of a protozoon nucleic acid. In some cases, methods herein result in enrichment of a cancer nucleic acid. In some cases, methods herein result in enrichment of a fetal nucleic acid.

### Use of Endonuclease/Exonuclease Combinations in Targeted Depletion

The method described herein for depleting a first nucleic acid results in a sequencing library with dramatically reduced complexity. Unwanted sequences are removed and the remaining sequences can be more readily analyzed by NGS techniques. The reduced complexity of the library can reduce the sequencer capacity required for clinical depth sequencing and/or reduce the computational requirement for accurate mapping of non-repetitive sequences or sequences of interest. The sequence that is enriched (e.g., relatively by depleting the unwanted or undesired sequences) can be searched in a bioinformatics database such as BLAST to determine the identity of the genes. The sequence information of the enriched nucleic acid can be used to determine the type of pathogen.

Through methods disclosed herein, a sample is treated so as to acquire exonuclease-protected ends, and then specific nucleic acids are cleaved so as to expose exonuclease-sensitive ends, such that a concurrent or subsequent exonuclease treatment selectively degrades nucleic acid cleavage products while leaving uncleaved, capped nucleic acids intact. Remaining nucleic acids are then used to prepare a sequencing library or otherwise assayed.

The various uses of the technology described above may include but are not limited to transplantation, cancer, infectious diseases, microbiome analysis, non-invasive prenatal testing (NIPT) and many others.

In some aspects, the method comprising the steps of denaturation of cfNA, hybridization to RS1 and RS2 random sequence adaptors coupled with the ligation of the hybridized single stranded cfNA inverted stubby adaptors terminal nucleotides thereby generating circular molecules comprising cfNA can be utilized to enrich short ssDNA or RNA, e.g., enriching mitochondrial or microbial cfNA, and library preparation. In some aspects, the enriched short sequences are typically about 100 nucleotides long or less than 100 nucleotides long. In some aspects, the methods described herein comprises an efficient ligation-based single-stranded library preparation method that is engineered to produce complex libraries in less than 24 h, less than 12 h, less than 10 h, less than 8 h or less than 6 h. In some aspects, the methods can be performed in 4 h. In some aspects, the methods can be performed in about 2.5 h or less. In some aspects, the methods can be performed from as little as 1 nanogram of input cfNA without alteration to the native ends of template molecules.

### Definitions

A partial list of relevant definitions is as follows.

As used herein, the term “enriched” is used in a relative sense, such that a second nucleotide or population comprising a second nucleotide is enriched upon the selective depletion of a first nucleotide or population comprising a first nucleotide. It does not need increase in an absolute sense to be enriched. Rather, an absolute increase or a relative increase resulting from depletion or deletion of other nucleic acids may constitute ‘enrichment’ as used herein.

As used herein, the term “deplete” or “depleting” is used in a relative sense, such that a first nucleotide or population comprising a first nucleotide is degraded upon the selective preservation of a second nucleotide or population comprising a second nucleotide. It does not need decrease in an absolute sense to be depleted. Rather, an absolute decrease or a relative decrease resulting from preservation of other nucleic acids may constitute ‘depleting’ as used herein.

As used herein, “about” a given value is defined as +/−10% of said given value including the absolute value of the given value.

As used herein, NGS or Next Generation Sequencing may refer to any number of nucleic acid sequencing technologies, such as 5.1 Massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, Single molecule real time (SMRT) sequencing, Tunnelling currents DNA sequencing, Sequencing by hybridization, Sequencing with mass spectrometry, Microfluidic Sanger sequencing, Microscopy-based techniques, RNAP sequencing, and In vitro virus high-throughput sequencing.

As used herein, to ‘modify’ a nucleic acid is to cause a change to a covalent bond in the nucleic acid, such as methylation, base removal, or cleavage of a phosphodiester backbone.

As used herein, to ‘direct transcription’ is to provide template sequence from which a specified RNA molecule can be transcribed.

“Amplified nucleic acid” or “amplified polynucleotide” includes any nucleic acid or polynucleotide molecule whose amount has been increased by any nucleic acid amplification or replication method performed in vitro as compared to its starting amount. For example, an amplified nucleic acid is optionally obtained from a polymerase chain reaction (PCR) which can, in some instances, amplify DNA in an exponential manner (for example, amplification to 2ⁿ copies in n cycles) wherein most products are generated from intermediate templates rather than directly from the sample template. Amplified nucleic acid is alternatively obtained from a linear amplification, where the amount increases linearly over time and which, in some cases, produces products that are synthesized directly from the sample.

The term “biological sample” or “sample” generally refers to a sample or part isolated from a biological entity. The biological sample, in some cases, shows the nature of the whole biological entity and examples include, without limitation, bodily fluids, dissociated tumor specimens, cultured cells, and any combination thereof. Biological samples come from one or more individuals. One or more biological samples come from the same individual. In one non limiting example, a first sample is obtained from an individual's blood and a second sample is obtained from an individual's tumor biopsy. Examples of biological samples include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free DNA, such as tumor DNA or fetal DNA. The samples include nasopharyngeal wash. Examples of tissue samples of the subject include but are not limited to, connective tissue, muscle tissue, nervous tissue, epithelial tissue, cartilage, cancerous or tumor sample, or bone. Samples are obtained from a human or an animal. Samples are obtained from a mammal, including vertebrates, such as murines, simians, humans, farm animals, sport animals, or pets. Samples are obtained from a living or dead subject. Samples are obtained fresh from a subject or have undergone some form of pre-processing, storage, or transport.

Nucleic acid sample as used herein refers to a nucleic acid sample for which the first nucleic acid is to be determined, A nucleic acid sample is extracted from a biological sample above, in some cases. Alternatively, a nucleic acid sample is artificially synthesized, synthetic, or de novo synthesized in some cases. The DNA sample is genomic in some cases, while in alternate cases the DNA sample is derived from a reverse-transcribed RNA sample.

“Bodily fluid” generally describes a fluid or secretion originating from the body of a subject. In some instances, bodily fluid is a mixture of more than one type of bodily fluid mixed together. Some non-limiting examples of bodily fluids include but are not limited to: blood, urine, bone marrow, spinal fluid, pleural fluid, lymphatic fluid, amniotic fluid, ascites, sputum, or a combination thereof.

“Complementary” or “complementarity,” or, in some cases more accurately “reverse-complementarity” refer to nucleic acid molecules that are related by base-pairing. Complementary nucleotides are, generally, A and T (or A and U), or C and G (or G and U). Functionally, two single stranded RNA or DNA molecules are complementary when they form a double-stranded molecule through hydrogen-bond mediated base paring. Two single stranded RNA or DNA molecules are said to be substantially complementary when the nucleotides of one strand, optimally aligned and with appropriate nucleotide insertions or deletions, pair with at least about 90% to about 95% or greater complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions and not stringent hybridization conditions. Hybridization temperatures are generally at least about 2° C. to about 6° C. lower than melting temperatures (Tₘ).

“Double-stranded” refers, in some cases, to two polynucleotide strands that have annealed through complementary base-pairing, such as in a reverse-complementary orientation.

“Known oligonucleotide sequence” or “known oligonucleotide” or “known sequence” refers to a polynucleotide sequence that is known. In some cases, a known oligonucleotide sequence corresponds to an oligonucleotide that has been designed, e.g., a universal primer for next generation sequencing platforms (e.g., Illumina, 454), a probe, an adapter, a tag, a primer, a molecular barcode sequence, an identifier. A known sequence optionally comprises part of a primer. A known oligonucleotide sequence, in some cases, is not actually known by a particular user but is constructively known, for example, by being stored as data accessible by a computer. A known sequence is optionally a trade secret that is actually unknown or a secret to one or more users but is known by the entity who has designed a particular component of the experiment, kit, apparatus or software that the user is using.

“Library” in some cases refers to a collection of nucleic acids. A library optionally contains one or more target fragments. In some instances the target fragments comprise amplified nucleic acids. In other instances, the target fragments comprise nucleic acid that is not amplified. A library optionally contains nucleic acid that has one or more known oligonucleotide sequence(s) added to the 3′ end, the 5′ end or both the 3′ and 5′ end. The library is optionally prepared so that the fragments contain a known oligonucleotide sequence that identifies the source of the library (e.g., a molecular identification barcode identifying a patient or DNA source). In some instances, two or more libraries are pooled to create a library pool. Libraries are optionally generated with other kits and techniques such as transposon mediated labeling, or “tagmentation” as known in the art. Kits are commercially available. One non-limiting example of a kit is the Illumina NEXTERA kit (Illumina, San Diego, CA).

The term “polynucleotides” or “nucleic acids” includes but is not limited to various DNA, RNA molecules, derivatives or combination thereof. These include species such as dNTPs, ddNTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA.

Phosphoramidates are the aliphatic amides of phosphoric acid and are widely employed in the synthesis of differentially protected phosphate esters as more stable alternatives to halophosphates. Phosphoramidate chemistry has been applied in the synthesis of nucleoside triphosphates.

The use of the terms upper strand and lower strands are arbitrary assigned to two strands of a double stranded polynucleotide as they appear in a diagrammatic point of view. As used herein an upper strand is typically used to denote the strand that comprises the 3′-X1ₘ-A-5′ and the 5′-B-X2ₙ-3′ is referred to as the “short stubby adapter”; and the bottom strand comprises the random sequences RS1 and RS2. It may be considered that for the purpose of this disclosure, the bottom strand comprises single stranded template regions (e.g., RS1 and RS2) for hybridization with cfNA.

The use of the term polynucleotide distinguishes a nucleic acid being described, as used herein, from an oligonucleotide, wherein the polynucleotide may comprise one or more oligonucleotides. An oligonucleotide is comprised of some, e.g. few nucleotides arranged in a single strand. A strand is used in the meaning known in common use of the term in the language. An oligonucleotide comprises 5, 6, 7, 8, 9, 10, 11, 12, 13, 14, 15 or about 20 or about 25 nucleotides. A polynucleotide may comprise one or more strands of oligonucleotides.

Before the present methods, compositions and kits are described in greater detail, it is to be understood that this invention is not limited to particular method, composition or kit described, as such may, of course, vary. It is also to be understood that the terminology used herein is for the purpose of describing particular embodiments only, and is not intended to be limiting, since the scope of the present invention will be limited only by the appended claims as construed herein. Examples are put forth so as to provide those of ordinary skill in the art with a more complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein are optionally used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method is contemplated to be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which may need to be independently confirmed.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Detection of Two Pathogens in a Human Biological Sample

An FFPE sample from a human was the source material to isolate two pathogens and detect a first pathogen P1 and a second pathogen P2. cfNA was isolated from the two samples. Selective depletion of abundant human sequences e.g., ribosomal sequences was first carried out by sequence guided cleavage and digestion. The sample is first subjected to heat denaturation at 90-95° C. for 10-30 minutes. The denatured cfNA is the mixed with a synthetic construct with the double stranded bidirectional short stubby adapters (derived from Illumina P5 and P7 bidirectional primers), flanked by single stranded random sequences at a temperature allowing hybridization. Circularization and low PCR amplification was carried on for 20 minutes, followed by ligation and digestion. The non-circularized material or single stranded circles are digested by exonuclease. The resultant circularized product is linearized by cleavage between the P5 and P7 5′-5′ juxtapositions and amplified using suitable primers. Primers binding to P5 and P7 elements can be used for further amplification and library generation.

### Example 2. Capturing Fragmented or Low Quality Genetic Material for Analysis

In this example, one exemplary generalized workflow is described for carrying out the features and methods of the invention for capturing fragmented or low quality DNA or RNA.

Synthetic partially double stranded nucleic acid construct: For the assay, synthetic double stranded construct is generated with a variety of RS1 and RS2 adapter sequences, the synthetic construct comprising a generalized structure: (i) a synthetic single oligonucleotide strand denoted by 3′-X1ₘ-A-5′-5′-B-X2ₙ-3′; where, X1ₘ and X2ₙ each denotes a sequence of m and n number of nucleotides respectively, wherein m and n depict any integer between 1 and 30, A and B each represent any nucleotide, wherein A and B are juxtaposed in 5′-5′ inverted orientation; where X1ₘ-A is a reverse primer and B-X2ₙ is a forward primer of a primer pair, having a 5′-5′ juxtaposed at A-B; (ii) a partially complementary synthetic oligonucleotide strand having a sequence denoted by 3′RS1-X1ₘ-A-5′-5′-B-X2ₙ-RS2-3′, as described in the previous sections. Each RS1 and RS2 adapter sequence comprises a molecular barcode. The 3′ ends of RS1 and RS2 are protected from ligation, or self-concatenation by replacing the 2′-deoxyribose at the 3′-end with a 2′,3′-dideoxyribose.

Denaturing, hybridizing and ligating sample cfNA: Cell free DNA was isolated from a biological sample. The sample is denatured at 90° C. for 5-30 minutes. The denatured sample is contacted with a synthetic construct comprising (i) a synthetic single oligonucleotide strand denoted by 3′-X1ₘ-A-5′-5′-B-X2ₙ-3′ and (ii) a partially complementary synthetic oligonucleotide strand having a sequence denoted by 3′RS1-X1ₘ-A-5′-5′-B-X2ₙ-RS2-3′, that exhibit Watson Crick nucleotide base pairing at 3′-X1ₘ-A-5′-5′-B-X2ₙ-3′, and single stranded regions at either end, e.g., the RS1 and RS2 regions, comprising adaptor sequences. The reaction mixture comprising the synthetic construct and the denatured cfNA are gradually cooled. As the reaction mixture is allowed to cool, the 3′-X1ₘ-A-5′-5′-B-X2ₙ-3′ strand and the 3′RS1-X1ₘ-A-5′-5′-B-X2ₙ-RS2-3′ hybridize, and the random sequences hybridize with the single stranded sequences present within the sample pool of cfDNA. In some cases, a single strand of cfNA sequence hybridizes at one end with an RS1 sequence, and with the corresponding RS2 sequence with the other end forming a circular/semicircular intermediate with the 3′ terminal nucleotides of 3′-X1ₘ-A-5′-5′-B-X2ₙ-3′ and the adjoining cfNA nucleotides at the respective sides from the hybridized cfNA portions that are not bonded. DNA ligation is performed to ligate the 3′ends of the 3′-X1ₘ-A-5′-5′-B-X2ₙ-3′ with the adjoining nucleotides from the hybridized sequences originating from the cfNA using a ligase capable of ligating nicked DNA/RNA, e.g. T4 RNA ligase 2, T4 DNA ligase, or splintR ligase. This results in a circular nucleic acid molecule.

Removal of 3′RS1-X1m-A-5′-5′-B-X2n-RS2-3′ strand: This strand comprising the adapter is removed by digestion. One exemplary method is using CRISPR guide RNA directed Cas9 nickase, which performs a single stranded nick, followed by nuclease digestion of the nicked linear strand. Optionally, any non-circularized nucleic acid material is digested, thereby reducing unwanted background material.

PCR amplification: The remaining strand of the synthetic construct now ligated to cfNA sequence fragments at the 3′ ends are subjected to limited cycle PCR amplification using primer sequences complementary to 3′-X1m-A-5′; and 5′-B-X2n-3′ respectively for generating short amplified sequences that comprise sequences from the cfNA sample. A size selection cleanup of the amplified DNA is performed.

Sequencing amplified DNA: The amplified product is then sequenced to identify the sequence of the fragment that hybridized each set of the RS1 and RS2 sequences carrying with it pieces of nucleic acid sequences originating from the cfNA.

Preparation of library: The amplified and sequenced elements corresponding to the previously unknown sequences originating from the cfNA can be cloned into a library of sequences, wherein the molecular barcodes encoded in the RS1 and RS2 sequences are used for identification of the cloned sequences.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein can be employed. It is intended that the following claims define the scope of the invention and that methods and structures within the scope of these claims and their equivalents be covered thereby.

What is claimed is:

1. A method of detecting the presence or absence of a target nucleic acid from a sample comprising a plurality of nucleic acid molecules, the method comprising:
denaturing a sample comprising the target nucleic acid,
contacting one or more nucleic acid molecules of the plurality of nucleic acid molecules with a synthetic nucleic acid comprising a first nucleic acid segment and a second nucleic acid segment that are in inverted orientation from each other,
generating one or more synthetic circularized nucleic acid molecules; and
sequencing the one or more synthetic circular nucleic acid molecules, thereby detecting the presence or absence of the target nucleic acid.

2. A method of amplifying a target nucleic acid from a sample comprising a plurality of nucleic acid molecules, the method comprising:
contacting one or more nucleic acid molecules of the plurality of nucleic acid molecules with a synthetic nucleic acid comprising a first nucleic acid segment and a second nucleic acid segment that are in inverted orientation from each other, wherein the one or more nucleic acid molecule comprises the target sequence, thereby generating one or more synthetic circularized nucleic acid molecules; and
amplifying the one or more synthetic circularized nucleic acid molecules, thereby amplifying the target nucleic acid.

3. A method of barcoding a plurality of nucleic acid molecules in a sample, the method comprising:
contacting the plurality of nucleic acid molecules with an a synthetic nucleic acid comprises a first nucleic acid segment and a second nucleic acid segment that are in inverted orientation from each other, wherein the first or the second nucleic acid segment comprises a molecular barcode,
generating one or more synthetic circular circularized nucleic acid molecules; wherein each synthetic circularized nucleic acid molecules comprises a nucleotide barcoding embedded within the circularized nucleic acid molecules.

4. The method of any one of the claims 1-3, wherein the synthetic nucleic acid is single stranded.

5. The method of any one of the claims 1-3, wherein the synthetic nucleic acid is double stranded, wherein the double stranded synthetic nucleic acid comprises single stranded regions.

6. The method of any one of the claims 1-5, wherein the synthetic nucleic acid comprises a sequence having a configuration: 3′-X1ₘ-A-5′-5′-B-X2ₙ-3′
wherein X1ₘ and X2ₙ each denotes a sequence of m and n number of nucleotides respectively, wherein m and n each is any integer between 1 and 30, and
A and B each represent any nucleotide, wherein A and B are juxtaposed in 5′-5′ inverted orientation.

7. The method of any one of the claims 1-6, wherein the sample is a biological sample.

8. The method of any one of the claims 1-7, wherein the biological sample comprises low quantity of the plurality of nucleic acid molecules, or low quality of the plurality of nucleic acid molecules or both.

9. The method of any one of the claims 1-7, wherein the biological sample comprises cell free nucleic acid (cfNA).

10. The method of any one of the claims 1-7, wherein the biological sample comprises frozen nucleic acid.

11. The method of any one of the claims 1-7, wherein the biological sample comprises ancient nucleic acid.

12. The method of any one of the claims 1-11, wherein the plurality of nucleic acid molecules comprise DNA.

13. The method of any one of the claims 1-11, wherein the plurality of nucleic acid molecules comprise RNA.

14. The method of any one of the claims 1-11, wherein the plurality of nucleic acid molecules is a mixture of DNA and RNA.

15. The method of any one of the claims 1-14, the plurality of nucleic acid molecules comprise single or double-stranded nucleic acid, or both.

16. The method of any one of the claims 1-15, further comprising a step of denaturing the plurality of nucleic acid molecules.

17. The method of any one of the claims 1-16, further comprising depleting one or more components of the plurality of nucleic acid molecules that is not bound to the synthetic nucleic acid.

18. The method of claim 17, wherein the depletion is performed before generation of a synthetic circularized nucleic acid molecules.

19. The method of claim 17, wherein the depletion is performed after generation of a synthetic circularized nucleic acid molecules.

20. The method of any one of the claims 17-19, wherein the depletion is performed using a nuclease.

21. The method of claim 20, wherein the nuclease is a DNA guided endonuclease.

22. The method of claim 21, wherein the nuclease is a DNA guided endonuclease is Argonaut (AGO).

23. The method of claim 20, wherein the nuclease is a CAS endonuclease.

24. The method of any one of the claims 1-23, further comprising annealing one or more adapter handles to the synthetic nucleic acid.

25. The method of claim 24, wherein an adapter handle is annealed to each termini of the single-stranded synthetic nucleic acid, the double stranded synthetic nucleic acid or a ligated product comprising the synthetic nucleic acid.

26. The method of claim 24 or 25, wherein an adapter handle comprises double stranded nucleic acid.

27. The method of any one of the claims 1-26, further comprising performing polymerase chain reaction.

28. The method of any one of the claims 1-27, further comprising incorporating one or more modifications in the synthetic circularized nucleic acid molecules.

29. The method of claim 28, wherein incorporating one or more modifications comprises incorporating a non-natural nucleotide, wherein the non-natural nucleotide is an LNA or a PNA.

30. The method of claim 28, wherein incorporating one or more modifications comprises incorporating a non-canonical nucleotide backbone linkage at the ligation point.

31. The method of claim 30, wherein the non-canonical nucleotide backbone linkage comprises an amide linkage, a triazole linkage, or a phosphoramidate.

32. The method of any of the claims 1-31, wherein the ends of the synthetic polynucleotide are not phosphorylated.

33. A method for selectively enriching one or more target nucleic acids comprising the method steps of any of the claims 1-32, wherein at least one or more nucleic acid components is depleted.

34. The method of claim 29, wherein the one or more nucleic acid components that is depleted is contaminant nucleic acid, microbial nucleic acid, host nucleic acid, ribosomal RNA, or repeat nucleic acid.

35. The method of any one of the claims 1-34, performed for diagnosing a disease.

36. The method of claim 35, wherein the disease is cancer.

37. The method of claim 35, wherein the disease is a microbial disease.

38. The method of claim 35, wherein the disease is a metabolic disease.

39. The method of claim 35, wherein the disease is genetic disease.

40. The method of any one of the claims 1-34 performed for a microbiome analysis.

41. The method of any one of the claims 1-34 performed for non-invasive prenatal testing.

42. A synthetic single or double-stranded nucleic acid comprising an oligonucleotide having a configuration: 3′-X1ₘ-A-5′-5′-B-X2ₙ-3′ wherein X1ₘ and X2ₙ each denotes a sequence of m and n number of nucleotides respectively, wherein m and n depict any integer between 1 and 30, A and B each represent any nucleotide, wherein A and B are juxtaposed in 5′-5′ inverted orientation.

43. The synthetic nucleic acid of claim 42, wherein the synthetic polynucleotide is double stranded, and wherein the double stranded polynucleotide comprises single stranded regions.

44. The synthetic nucleic acid of claim 42, wherein the single stranded regions within the double stranded polynucleotide comprise a sequence of 3 or more random nucleotides at the 5′ or the 3′ end of the double stranded region or both.

45. A nucleic acid library comprising the synthetic circularized nucleic acid molecule or portions thereof, or derivatives thereof of any one of the claims 1-44.
